(19) United States
(12) Patent Application Publication
(10) Pub. No.: US 2026/0237455 A1
(43) Pub. Date: Aug. 13, 2026

(54) UTILIZING A DIGITAL PHENOMAP AND GERMLINE PATIENT DATA TO GENERATE A MEASURE OF TARGET DISCOVERY POWER AND GENE TARGETS

(71) Applicant: Recursion Pharmaceuticals, Inc., Salt Lake City, UT (US)

(72) Inventors: Daniel Patrick MALJOVEC, Holladay, UT (US); Hayley Jeton DONNELLA, Austin, TX (US); Imran Saeedul HAQUE, Salt Lake City, UT (US); Ryan Patrick SMITH, Salt Lake City, UT (US); Ryan Lawton SUBARAN, Salt Lake City, UT (US); William Paul BONE, Salt Lake City, UT (US); Xin WANG, San Francisco, CA (US)

(21) Appl. No.: 19/050,504

(22) Filed: Feb. 11, 2025

(51) Int. Cl. G16B 20/00; G16B 45/00; G16H 10/60; G16H 50/70

(52) U.S. Cl. CPC G16B 20/00; G16B 45/00; G16H 10/60; G16H 50/70

(57) ABSTRACT

The present disclosure relates to systems, non-transitory computer-readable media, and methods that utilize a phenomap and germline data to generate a measure of target discovery power and gene targets for trait of interest. Indeed, the disclosed systems can sample a test subset of genomic patient data samples from a combined set of data corresponding to a trait of interest. For instance, the disclosed systems identify a test gene target from the sampled test subset that satisfies a threshold correlation with the trait of interest. In some instances, the disclosed systems generate a test phenomap gene target by comparing a test gene target in the digital phenomap with additional genes in the digital phenomap. Moreover, the disclosed systems can generate a measure of target discovery power of the digital phenomap by comparing the test phenomap gene target with a gene target set identified from the combined set.

### BACKGROUND

Recent years have seen significant developments in hardware and software platforms that utilize computational models to identify relationships between genes for drug discovery purposes. For example, conventional systems utilize computing devices to parse through volumes of gene data to identify potential relationships. Despite recent advancements, conventional systems continue to experience a variety of technical problems, including accuracy, efficiency, and operational flexibility of implementing computing devices in discovering gene relationships from patient data.

### SUMMARY

Embodiments of the present disclosure provide benefits and/or solve one or more of the foregoing or other problems in the art with systems, non-transitory computer-readable media, and methods for utilizing a digital phenomap and germline patient data to generate a measure of target discovery power for the digital phenomap and gene targets for an inference-time trait of interest. For example, in one or more implementations, the disclosed systems sample from a test subset of genomic patient data samples (e.g., a subset of a combined set of genomic patient data for a trait of interest of a trait class). Specifically, the disclosed systems identify a test gene target (e.g., the test gene target satisfies a threshold correlation with the trait of interest) from the sampled test subset. Further, in one or more implementations, the disclosed systems generate a test phenomap gene target by using a digital phenomap. Moreover, in one or more implementations, the disclosed systems generate a measure of target discovery power of the digital phenomap by comparing the test phenomap gene target with a gene target set identified from the combined set of genomic patient data.

Additional features and advantages of one or more embodiments of the present disclosure are outlined in the description which follows, and in part will be obvious from the description, or may be learned by the practice of such example embodiments.

### BRIEF DESCRIPTION OF THE DRAWINGS

The detailed description provides one or more embodiments with additional specificity and detail through the use of the accompanying drawings, as briefly described below.

FIG. 1 illustrates an overview diagram of a phenomap power discovery system 100 leveraging germline patient data and a digital phenomap to generate gene target(s) and a measure of target discovery power in accordance with one or more embodiments.

FIG. 2 illustrates an example diagram of the phenomap power discovery system 100 generating a digital phenomap from cells exposed to perturbations in accordance with one or more embodiments.

FIG. 3 illustrates an example diagram of using patient genomics to identify gene targets in accordance with one or more embodiments.

FIG. 4 illustrates an example diagram of the phenomap power discovery system generating a measure of target discovery power for a digital phenomap from a test subset of genomic patient data samples in accordance with one or more embodiments.

FIG. 5 illustrates an example diagram of the phenomap power discovery system generating a measure of target discovery power for a digital phenomap from an additional test subset of genomic patient data samples in accordance with one or more embodiments.

FIG. 6 illustrates an example diagram of the phenomap power discovery system generating a test phenomap gene target by comparing feature vectors within a digital phenomap in accordance with one or more embodiments.

FIG. 7 illustrates an example diagram of the phenomap power discovery system generating a measure of target discovery power for a first trait class and an additional measure of target discovery power for a second trait class in accordance with one or more embodiments.

FIG. 8 illustrates an example diagram of the phenomap power discovery system receiving a target discovery power query for an inference-time trait of the trait class in accordance with one or more embodiments.

FIG. 9A illustrates an example diagram of the phenomap power discovery system generating measures of target discovery power for a test subset and an additional test subset and providing the measures to a client device in accordance with one or more embodiments.

FIG. 9B illustrates an example diagram of the phenomap power discovery system generating a target discovery power prediction based on measures of target discovery power in accordance with one or more embodiments.

FIG. 10 illustrates an example diagram of the phenomap power discovery system generating an inference-time phenomap gene target from inference-time patient data samples and providing the inference-time phenomap gene targets to a client device in accordance with one or more embodiments.

FIG. 11 illustrates experimental results of the phenomap power discovery system generating phenomap gene targets from subsets of patient data samples and further confirming the phenomap gene targets as accurate based on comparing the phenomap gene targets with ground truth gene targets obtained from a full dataset in accordance with one or more embodiments.

FIG. 12 illustrates an example environment of the phenomap power discovery system in accordance with one or more embodiments.

FIG. 13 illustrates an example series of acts to generate a measure of target discovery power of a digital phenomap in accordance with one or more embodiments.

FIG. 14 illustrates a block diagram of a computing device for implementing one or more embodiments.

### DETAILED DESCRIPTION

Embodiments of the present disclosure provide benefits and/or solve one or more of the foregoing or other problems in the art with systems, non-transitory computer-readable media, and methods of a phenomap power discovery system that utilizes genomics datasets to estimate the power increase of digital phenomaps relative to a trait class (e.g., identify genetic signatures at relatively smaller patient sample sizes). Indeed, in one or more embodiments, the phenomap power discovery system uses digital phenomic maps to recover otherwise unsalvageable signals (e.g., map expansion) from genomics datasets and estimates the likelihood of finding hits for particular trait classes (e.g., endocrinology, neurology, immunology, etc.). For example, the phenomap power discovery system can utilize a subsampling approach to estimate the power of a digital phenomap in identifying genes pertinent to a particular trait (and how many samples would be needed to detect signals for future traits).

To illustrate, given a trait of interest (e.g., type II diabetes), the phenomap power discovery system can collect a random subsample (e.g., 10,000 samples) from a larger genetics database (e.g., having 1 million samples). Moreover, the phenomap power discovery system can perform a genetic association analysis for the random subsample to identify one or more subsampled significant genes for the trait. Further, the phenomap power discovery system can also perform a digital phenomap analysis to expand on the subsampled significant genes from the random subsample. For example, the phenomap power discovery system can utilize the phenomap to identify predicted similar genes from the subsampled significant genes (e.g., the phenomap power discovery system identifies genes in the feature vector space of the digital phenomap that are similar to the subsampled significant genes from the random subsample).

Additionally, the phenomap power discovery system can perform a full genetic association analysis using the full genetics database to identify significant genes (e.g., ground truth genes to determine how powerful the phenomap is). Moreover, the phenomap power discovery system can compare the predicted similar genes (e.g., the test phenomap gene target(s) 110 generated from the digital phenomap 104 from a random subsample) relative to the significant genes (e.g., a gene target set acting as the ground truth measures) identified from the full data set. In this manner, the phenomap power discovery system can measure the power of the digital phenomap in identifying significant genes for a trait class.

Moreover, by repeatedly performing this subsampling approach with different subsamples (e.g., 1,000, 2,000, 5,000 samples) relative to different types of traits of interest for the same trait class and across different phenomaps of biology, the phenomap power discovery system can estimate the power of different phenomaps in accurately identifying significant genes for a particular trait class (e.g., endocrinology, immunology, etc.).

Furthermore, the phenomap power discovery system can use the measures of target discovery power information to make inferences or predictions about digital phenomaps for future data sets (e.g., inference-time datasets). Indeed, given a data set for a particular trait class, the phenomap power discovery system can utilize digital phenomaps to identify similar genes of interest in an inference-time data sample. Moreover, the phenomap power discovery system can estimate a target discovery power prediction of different digital phenomaps and the likelihood of identifying one or more hits in an inference-time data sample using the digital phenomaps. Moreover, the phenomap power discovery system can also estimate the likelihood of finding additional hits through gathering additional data (e.g., a target sample size different than an initial sample size of an inference-time data sample). In other words, the phenomap power discovery system can estimate a quantity to increase experimentation by (e.g., for inference time samples) in order to discover additional gene targets without using a digital phenomap.

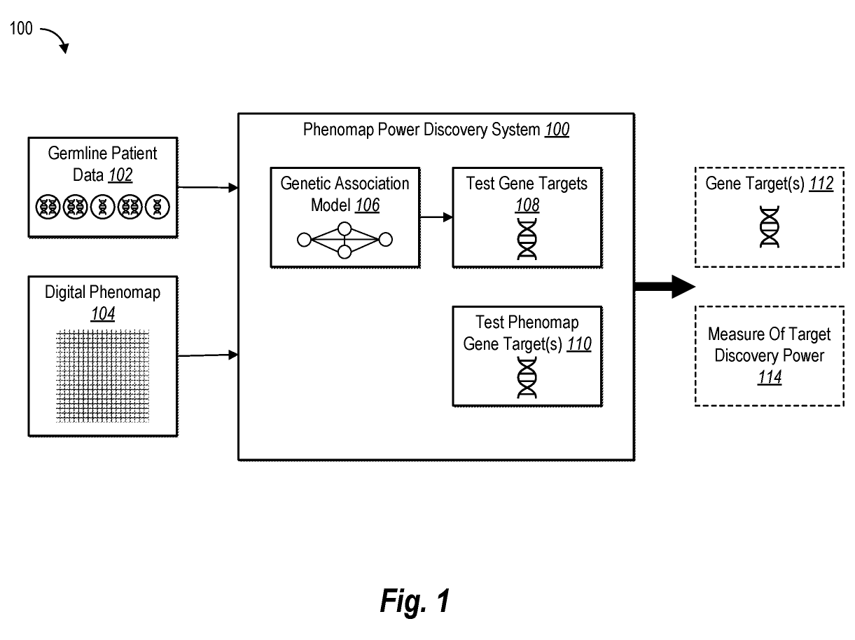

FIG. 1 illustrates a phenomap power discovery system 100 generating gene target(s) and/or measures of target discovery power based on germline patient data and a digital phenomap in accordance with one or more embodiments. Specifically, the phenomap power discovery system 100 generates a measure of target discovery power 114 of a digital phenomap 104 relative to a trait class and also can generate gene targets for a trait of interest (e.g., an inference-time trait of interest) of a trait class.

As used herein, the term “trait class” refers to a group, classification, or related set of traits (e.g., related genetic traits that correspond to similar diseases, expressions, or biological pathways). Specifically, a trait class includes overlapping or related biological domains (e.g., for purposes of drug discovery). For example, a trait class refers to categories such as immunology, metabolism, cardiology, endocrinology, neurology, and nephrology.

For instance, for a trait class of endocrinology, the trait class can encompass biological mechanisms spanning from hormone production, signaling and various hormone’s effects on maintaining homeostasis (e.g., balance). Indeed, the trait class of endocrinology covers multi-system processes of a biological subject and conditions such as type II diabetes, thyroid disorders, and hormonal imbalances.

Further, for a trait class of immunology, the trait class encompasses biological mechanisms spanning immune response, regulation, and interaction of various physiological systems. Specifically, the trait class of immunology covers various roles of an immune system in defending against pathogens, inflammation, and autoimmune disorders (e.g., arthritis).

As used herein, the term “trait of interest” refers to a specific characteristic, condition, or trait within a trait class (e.g., a genetic trait that corresponds to a particular disease or expression). For instance, a trait of interest includes a medical characteristic/condition resulting from (at least in part) a genetic component. For instance, traits of interest can include type II diabetes, pituitary disorders, asthma, blue eyes, reaching the age of one hundred (e.g., centenarian), having flat feet, and/or arthritis. Moreover, various clinical studies can collect biological data (e.g., genomic data) from patients that manifest a trait of interest.

As shown in FIG. 1, the phenomap power discovery system 100 processes germline patient data 102 and a digital phenomap 104. For instance, the germline patient data 102 refers to inheritable biological information. As used herein, the term “germline patient data” refers to biological data (e.g., of human subjects) involved in reproduction. In contrast with somatic cells (which do not participate directly in reproduction and are not passed on to offspring), the germline patient data 102 undergoes meiosis during the reproduction process. Specifically, the germline patient data 102 carries genetic information that is passed to the next generation through fertilization.

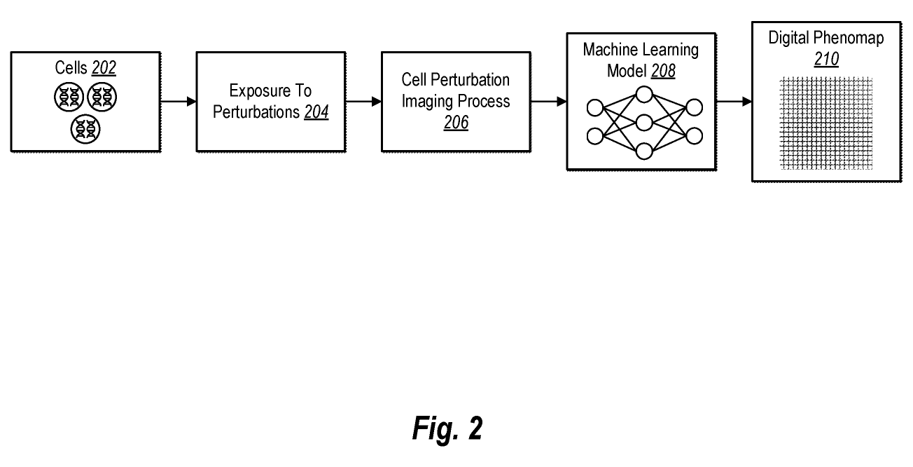

As used herein, the term “digital phenomap” refers to collection of data regarding phenotypes resulting from perturbation (e.g., a map of biology that allows for comparisons of phenotypes resulting from cellular perturbations). In particular, a phenomap can include a plurality of embeddings from phenotypes resulting from these perturbations. To illustrate, the phenomap power discovery system 100 can apply perturbations to cells and capture digital images of the perturbed cell phenotypes. The phenomap power discovery system 100 can utilize a machine learning model to generate phenomic embeddings from these digital images and combine these embeddings to generate a digital phenomap 210. Indeed, in some implementations the digital phenomap includes a collection of embeddings for a plurality of phenomic digital images that allows for comparisons of the underlying perturbations. For example, the phenomap power discovery system 100 can determine a variety of similarity metrics between these embeddings (e.g., cosine similarity or distance metrics) to determine relationships between perturbations. Thus, the digital phenomap 104 can include embeddings and/or a mapping of relationships/similarity between perturbations and phenotypic traits. Additional details regarding generating a digital phenomap are provided below (e.g., in relation to FIG. 2).

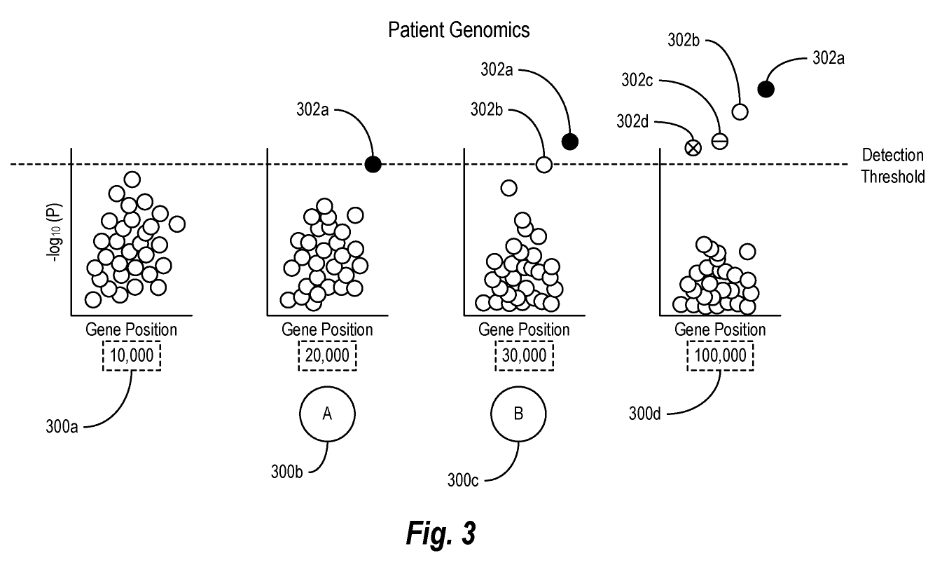

As shown in FIG. 1, the phenomap power discovery system 100 uses a genetic association model 106 to process the germline patient data 102 and generate test gene targets 108. More details regarding the phenomap power discovery system 100 using the genetic association model 106 to generate the test gene targets 108 is given below in FIG. 3. Furthermore, FIG. 1 also shows that based on the test gene targets 108 and test phenomap gene target(s) 110 (e.g., identified from the digital phenomap 104), the phenomap power discovery system 100 can generate gene target(s) 112 and/or a measure of target discovery power 114. For instance, the measure of target discovery power 114 is a measure of power relative to the digital phenomap 104 for a trait class.

FIG. 1 shows the gene target(s) 112 and the measure of target discovery power 114 in dotted lines because the phenomap power discovery system 100 can optionally perform these actions. For instance, during a testing phase, the phenomap power discovery system 100 generates the measure of target discovery power 114 to score the digital phenomap 104 relative to the trait class. Further, during inference-time, the phenomap power discovery system 100 generates the gene target(s) 112 based on the digital phenomap 104. Accordingly, FIG. 1 generally shows the phenomap power discovery system 100 leveraging both germline patient data 102 and the digital phenomap 104 to test the power of the digital phenomap 104 and to further generate the gene target(s) 112 for inference-time data samples (e.g., germline patient data).

As mentioned above, conventional systems suffer from a variety of deficiencies related to accuracy, efficiency, and operational flexibility. For example, conventional systems suffer from computational inaccuracies due to real-world constraints on accessibility of data samples and time. Specifically, conventional systems attempt to identify related genes for drug discovery purposes by sampling and applying models from patient data sets for a trait of interest. However, due to a variety of computational limitations (e.g., resources, time, data sparsity), conventional systems are typically limited in their ability to accurately extract gene relationships from genomic dataset. To illustrate, conventional systems typically have data size requirements that are prohibitively large (millions of patient samples). For example, it is often impractical or impossible to ascertain multi-million person biobanks with high quality phenotype data across human disease in order to accurately identify gene targets through conventional data analysis models and computational systems..

As a result, conventional systems suffer from either inaccurately finding genetic associations (e.g., for a trait of interest) or failing to find any genetic associations (e.g., due to the small size of data samples). To illustrate, for a centenarian study (e.g., patients that are over one hundred years of age), conventional systems typically are limited in their ability to collect genetic data from these subjects. For instance, subjects that are over 100 years old typically are unavailable or very limited in number. Thus, conventional systems are faced with the challenge of working with a relatively small sample size of centenarians and struggle to identify related gene targets.

Furthermore, conventional systems suffer from computational inefficiencies. For example, as just discussed conventional systems typically require extensive computational resources to collect sufficient data samples to accurately determine genetic associations. Moreover, conventional systems that do manage to collect a substantial amount of data (for drug discovery purposes of a trait of interest) often spend an exorbitant amount of resources, time, and computing power to analyze, process, and store the data.

In addition, conventional systems are further faced with challenges of dealing with unsalvageable data signals. Specifically, conventional systems attempt to collect data samples to study a trait of interest, however, many of the data samples contain signals that are unintelligible or seemingly irrelevant. Thus, conventional systems further face the issue of inefficiently collecting data (that contains unsalvageable signals) and potentially relying on unsalvageable signals to perform data analysis.

In addition, conventional systems suffer from operational inflexibilities. For example, conventional systems are typically limited to relying on existing datasets for specific traits of interest to identify genetic associations. In other words, conventional systems are operationally limited in how they can collect and analyze data to generate predicted gene targets. For instance, conventional systems typically have to resort to collecting as many data samples as possible to increase their chance of finding genetic associations for a trait of interest utilizing existing modeling approaches. Moreover, conventional approaches are unable to predict the number or type of samples needed to identify a gene target for a particular trait of interest.

In one or more embodiments, the phenomap power discovery system 100 improves computational accuracy relative to conventional systems. For example, the phenomap power discovery system 100 leverages the germline patient data 102 (e.g., genomic patient data samples) and digital phenomaps to more accurately hone in on gene targets for a trait of interest. In other words, even with the limitations of gathering data for specific traits of interest (e.g., centenarian studies), the phenomap power discovery system 100 is able to identify gene targets that would not otherwise be able to be identified by using the digital phenomap 104 to identify gene targets. Specifically, the phenomap power discovery system 100 improves the discovery power of finding gene targets for a trait of interest (e.g., of a certain sample size) by leveraging the digital phenomap 104. For instance, the phenomap power discovery system 100 uses a genetic association model to find one or more gene targets from a genomic patient data samples (e.g., that satisfy a threshold correlation with a trait of interest) and then determines which additional genes in the digital phenomap are close (e.g., via cosine similarity) to the gene target identified from the test subset.

In addition, the phenomap power discovery system 100 can generate a predicted improvement in discovery power prior to utilizing a phenomap for a particular trait of interest at inference time. Specifically, the phenomap power discovery system 100 can generate a measure of discovery power based on a sub-sampling technique for existing germline datasets to infer discovery power for other traits of interest. To illustrate, the phenomap power discovery system 100 initially generates the measure of target discovery power 114 for the digital phenomap 104 by using a test subset of genomic patient data samples to identify test gene targets and further generate test phenomap gene targets from the digital phenomap. Moreover, the phenomap power discovery system 100 can compare the test phenomap gene targets to a ground truth (e.g., set of gene targets in a full dataset) to determine a measure of power of the digital phenomap 104.

In doing so, the phenomap power discovery system 100 (e.g., by leveraging the digital phenomap 104) generates target discovery predictions for how much the digital phenomap 104 will boost an inference-time dataset. Thus, at inference-time, the phenomap power discovery system 100 not only improves the accuracy of finding additional gene targets for a trait of interest despite the real-world limitations on resources, time, and rarity of certain traits of interest; the phenomap power discovery system 100 also provides improved predictions of the degree or extent to which a phenomap will enhance the ability to identify additional gene targets from any particular dataset. The phenomap power discovery system 100 can also predict the samples needed to identify a gene target for particular trait of interest utilizing one or more phenomaps.

In one or more embodiments, the phenomap power discovery system 100 improves computational efficiency relative to conventional systems. Indeed, as mentioned above, despite limitations in sample size (e.g., 10K sample size), the phenomap power discovery system 100 can identify gene targets from the digital phenomap 104 that conventional systems would otherwise deem insignificant. Moreover, the phenomap power discovery system 100 generates the measure of target discovery power 114 for a specific trait class (e.g., endocrinology) and at inference-time, the phenomap power discovery system 100 can generate a target discovery power prediction for an inference-time trait of interest of the same trait class. In other words, the phenomap power discovery system 100 can determine how much a digital phenomap can boost a genomic patient data sample to find additional gene targets that are associated with a trait of interest. In this manner, the phenomap power discovery system 100 allows for more informed, efficient utilization of any particular phenomap. Indeed, the phenomap power discovery system 100 can select and utilize an appropriate phenomap for a particular inquiry with advanced knowledge of the power/likelihood of using the phenomap to discover a particular gene target relative to an input germline database. This results in fewer wasted resources in applying phenomaps inefficiently to trait classes that fail to align with the strengths of a particular phenomap while also improving the efficacy in utilizing each phenomap for a particular trait of interest.

Moreover, in some embodiments, the phenomap power discovery system 100 can further improve efficiency by generating a gene target discovery likelihood metric that indicates that increasing a genomic patient data sample by a threshold amount can further boost the likelihood of identifying additional gene targets. In other words, the phenomap power discovery system 100 can intelligently inform an inference-time client device regarding an efficient sample size. In doing so, the phenomap power discovery system 100 can save resources by indicating a threshold sample size (e.g., such as increasing the sample size to 20K to efficiently discover additional gene targets for an inference-time trait of interest). Thus, the phenomap power discovery system 100 can save resources, time, and computing power to analyze, process, and collect the data for discovering gene targets.

Relatedly, the phenomap power discovery system 100 further improves upon operational flexibility. As discussed above, the phenomap power discovery system 100 allows for inference-time client devices to discover gene targets in the digital phenomap 104 even with limited data sample sizes. Moreover, as also discussed, the phenomap power discovery system 100 predicts a target discovery power of an inference-time dataset for a client device and can further indicate how much to increase a sample size by to discover additional gene targets. Moreover, the phenomap power discovery system 100 brings together two distinct sets of data (e.g., genomic patient data samples and the digital phenomap 104) to enhance the ability of drug discovery processes in identifying genetic relationships. Thus, due to the accuracy and efficiency improvements of the phenomap power discovery system 100, the phenomap power discovery system 100 also is more operationally flexible in identifying genetic relationships for inference-time traits of interest.

As mentioned above, the phenomap power discovery system uses machine learning methods to generate a digital phenomap that includes embeddings or feature vectors for different gene perturbations. As shown in FIG. 2, the phenomap power discovery system 100 generates a digital phenomap from cells in accordance with one or more embodiments. As used herein, the term “cell” refers to a structural, functional, and biological unit of living organisms. Specifically, a cell can vary in size, shape, and function depending on the organism and the role of the cell. For example, a cell can include a plasma membrane to separate the internal cell environment from the external surroundings and the cell can further contain genetic material. .

As shown in FIG. 2, the phenomap power discovery system 100 performs an act 204 of exposing the cells 202 to perturbations. As used herein, the term “perturbation” (e.g., cell perturbation) refers to an alteration or disruption to a cell or the cell’s environment (to elicit potential phenotypic changes to the cell). In particular, the term perturbation can include a gene perturbation (i.e., a gene-knockout perturbation) or a compound perturbation (e.g., a molecule perturbation or a soluble factor perturbation). A compound can include a small molecule (e.g., a pharmaceutical compound/drug including alkaloids, antibiotics, steroids, vitamins, NSAIDs, among others). A compound can also include large molecule (e.g., proteins, antibodies, nucleic acids, polysaccharides, glycoproteins, among others). These perturbations are accomplished by performing a perturbation experiment. A perturbation experiment refers to a process for a perturbation to a cell. A perturbation experiment also includes a process for developing/growing the perturbed cell into a resulting phenotype.

As shown in FIG. 2, the phenomap power discovery system 100 further performs an act 206 of a cell perturbation imaging process. As used herein, the term perturbation imaging process (or generating phenomic digital images), refers to the phenomap power discovery system 100 generating a digital image portraying a cell exposed to a perturbation (e.g., a cell after applying a perturbation). For example, a perturbation image includes a digital image of a cell after application of a perturbation and further development of the cell. Thus, a perturbation image comprises pixels that portray a modified cell phenotype resulting from a particular cell perturbation.

Furthermore, the phenomap power discovery system 100 embeds the perturbation images into a low dimensional feature space via a machine learning model (e.g., a convolutional neural network) to generate perturbation image embeddings (e.g. feature vectors). Thus, a perturbation embedding includes a feature vector generated by application of various convolutional neural network layers (at different resolutions/dimensionality).

As shown, in FIG. 2, the phenomap power discovery system generates the perturbation image embeddings using a machine learning model 208. In one or more embodiments a machine learning model includes a computer algorithm or a collection of computer algorithms that can be trained and/or tuned based on inputs to approximate unknown functions. For example, a machine learning model can include a computer algorithm with branches, weights, or parameters that changed based on training data to improve for a particular task. Thus, a machine learning model can utilize one or more learning techniques to improve in accuracy and/or effectiveness. Example machine learning models include various types of decision trees, support vector machines, Bayesian networks, random forest models, or neural networks (e.g., deep neural networks).

As used herein, the term “perturbation embedding” (or individual perturbation embeddings, individual perturbation image embeddings or phenomic image embeddings) refers to a numerical representation of a perturbation image resulting from a perturbation to a cell. For example, an individual perturbation embedding includes a feature vector representation of a perturbation image generated by a machine learning model (e.g., a convolutional neural network or other machine learning embedding model). Thus, an individual perturbation embedding includes a feature vector generated by application of various convolutional neural network layers (at different resolutions/dimensionality). In other words, the individual perturbation embedding represents in a numerical format the phenotypic traits of an image of a perturbed cell.

The phenomap power discovery system 100 can utilize a variety of models to generate the perturbation embeddings. For example, in one or more embodiments, the phenomap power discovery system 100 utilizes internal feature vectors of a convolutional neural network trained to predict perturbations to generate the perturbation embeddings. Moreover, in some embodiments, the phenomap power discovery system 100 utilizes a masked autoencoder to generate the perturbation embeddings. To illustrate, the phenomap power discovery system 100 can utilize the models as described in UTILIZING MACHINE LEARNING AND DIGITAL EMBEDDING PROCESSES TO GENERATE DIGITAL MAPS OF BIOLOGY AND USER INTERFACES FOR EVALUATING MAP EFFICACY, US Patent App. No. 18/392,989, filed December 21, 2023, microscopy representation autoencoder models as described in UTILIZING MASKED AUTOENCODER GENERATIVE MODELS TO EXTRACT MICROSCOPY REPRESENTATION AUTOENCODER EMBEDDINGS, US Patent App. No. 18/545,399, filed December 19, 2023, which are incorporated by reference herein in their entirety.

As shown, the phenomap power discovery system 100 can utilize these embeddings to generate a digital phenomap 210. For instance, the phenomap power discovery system 100 perturbs genes involved in meiosis, genes involved in cell development, genes involved in fertility and gamete function, gene regulating chromosome segregation, genes involved in epigenetic regulation, and/or perturbations involving environmental or chemical perturbations (e.g., hormonal perturbations, oxidative stress, chemicals/drugs) to determine how the environmental/chemical perturbations effect cell development. To illustrate, the digital phenomap 210 includes a first dimension for the feature vectors of different groups of cells and a second dimension for the different perturbations (e.g., that target one or more specific genes or groups of genes in a pathway). As discussed above, for each perturbation to a cell or a group of cells, the phenomap power discovery system 100 generates a phenomic image (e.g., via the imaging process), and then further utilizes a machine learning model to generate an embedding representation of the phenomic image which is stored in a matrix cell of the digital phenomap 210.

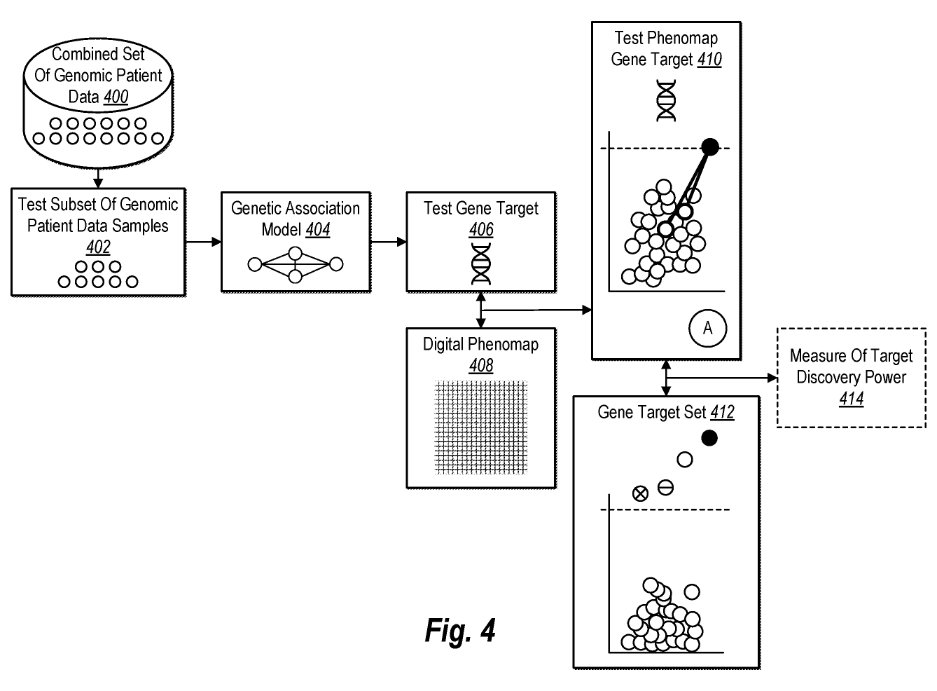

As mentioned above, without phenomaps, patient genomics can include analyzing large volumes of genomic data utilizing various models to identify potential gene targets. The phenomap power discovery system 100 can leverage patient genomics with phenomics data to identify gene targets. FIG. 3 illustrates utilizing patient genomics to identify genetic relationships for a specific trait of interest of a trait class (without phenomaps) and FIG. 4 illustrates utilizing patient genomics with a phenomap to identify additional gene targets. FIG. 3 illustrates example progression of a genomic dataset as samples are gathered and analyzed. In particular, the phenomap power discovery system 100 at a first time accesses a genomic dataset that includes a sample size 300a of 10K samples. As shown in FIG. 3, the 10K sample size is too small for to discover gene targets (without phenomic information). In other words, without phenomics, this sample size unable to yield significant gene targets for a trait of interest (at the sample size 300a of 10K). As shown in FIG. 3, each of the graphs show an x axis of gene and a y axis of -log₁₀ (P). Specifically, the graph illustrates that for a gene at a specific position in the genome, the patient genomics can yield a p value or a significance score of a gene relative to the sample size of data for the trait of interest (e.g., accordingly the subsets of patient data shown in FIG. 3 is for a specific gene at a specific genomic position, and as the sample size increases, the significance or p-value of the genes at the specific position in the genome changes).

FIG. 3 also illustrates additional sampling to a sample size 300b (e.g., represented as A) with a sample size of 20K. Specifically, FIG. 3 shows the phenomap power discovery system 100 (without a phenomap) identifying a first test gene target 302a (e.g., a first gene signal) from the sample size 300b of 20K samples. For instance, FIG. 3 shows the utilizing patient genomics to identify the first test gene target 302a. In other words, the phenomap power discovery system 100 identifies (without a phenomap) the first test gene target 302a as satisfying a statistical significance threshold relative to the sample size 300b of the trait of interest.

Moreover, FIG. 3 illustrates additional sampling to a sample size 300c (e.g., represented as B) with a sample size of 30K. Specifically, FIG. 3 shows the phenomap power discovery system 100 (without a phenomap) identifying the first test gene target 302a and a second test gene target 302b (e.g., a second gene signal) from the sample size 300c of 30K samples. For instance, FIG. 3 shows using patient genomics to identify both test gene targets as satisfying a statistical significance threshold relative to the sample size 300c of the trait of interest.

Furthermore, FIG. 3 shows a sample size 300d for a sample size of 100K (e.g., the full dataset, in other words the combined set of genomic patient data) and using patient genomics, the phenomap power discovery system 100 identifies a set of gene targets 302a-302d. Specifically, the phenomap power discovery system 100 can use patient genomics to perform a genetic association analysis and identify genes from a plurality of patient genomes that exhibit significant correlation (e.g., a third test gene target 302c and a fourth test gene target 302d in addition to the first test gene target 302a and the second test gene target 302b) with the specific trait of interest.

As mentioned above, the phenomap power discovery system 100 can further improve upon patient genomics by leveraging a digital phenomap to identify gene targets. In one or more embodiments, the phenomap power discovery system 100 performs two processes, a first process of measuring target discovery power for digital phenomaps (e.g., relative to a trait class) and a second process of inferring a prediction of a measure of discovery power of a digital phenomap for an inference-time trait of interest (e.g., of the same trait class). FIG. 4 illustrates the first process, specifically, the phenomap power discovery system 100 generating a measures of target discovery power based on a test subset of genomic patient data samples in accordance with one or more embodiments.

FIG. 4 illustrates the phenomap power discovery system 100 generating a measure of target discovery power for a given digital phenomap. In other words, the phenomap power discovery system 100 can determine whether a digital phenomap is appropriate for a particular phenotype (e.g., trait class). In other words, a lower measure of target discovery power can indicate that a digital phenomap is inappropriate (e.g., the digital phenomap lacks reliable predictive power relative to a particular phenotype) for a particular phenotype. While a higher measure of target discovery power can indicate that a digital phenomap is appropriate (e.g., possesses reliable predictive power relative to a particular phenotype) for a particular phenotype.

FIG. 4 shows a combined set of genomic patient data 400 (e.g., a full dataset) and the phenomap power discovery system 100 sampling a test subset 402 of genomic patient data samples from the full dataset. As used herein, the term “combined set of genomic patient data” refers to a dataset from which subsets of data are sampled or drawn. For example, a combined set of genomic patient data includes an over-arching, consolidated, complete or full dataset of genomic patient data (from which different subsets of data are drawn). Specifically, for a particular trait of interest (e.g., type II diabetes), the phenomap power discovery system 100 obtains the combined set of genomic patient data 400 that includes patients that exhibit the particular trait of interest and their corresponding genomic data. In other words, the combined set of genomic patient data 400 contains a plurality of genomes for patients that exhibit the particular trait of interest. Accordingly, the phenomap power discovery system 100 uses the combined set of genomic patient data 400 to identify genetic associations between different patients and to further identify genomic patterns and commonalities amongst patients with the same trait of interest (e.g., type II diabetes). To illustrate, the combined set of genomic patient data 400 (e.g., the full dataset) can contain one hundred thousand samples of patient data (e.g., one hundred thousand genomes for the particular trait of interest), similar to the full dataset shown above in FIG. 3 of the sample size 300d.

As mentioned, the phenomap power discovery system 100 samples the test subset 402 of genomic patient data samples from the full dataset. In one or more embodiments, a “test subset of genomic patient data samples” refers to a subsample of a dataset (e.g., a subset of the combined set of genomic patient data for a particular trait of interest). In particular, if the full dataset contains one million samples of patient data, the test subset of genomic patient data samples can contain ten thousand, or any variation of a sub-portion of the full dataset (e.g., 20K, 30K, 100K, etc.). For example, to generate the measure of target discovery power of a digital phenomap, the phenomap power discovery system 100 initially samples a test subset of the full dataset (e.g., the combined set of genomic patient data) to determine the power of the map at the specific sample size of the test subset of genomic patient data samples. More details are provided below.

For purposes of illustration in FIG. 4, the phenomap power discovery system 100 samples the test subset 402 that corresponds with (A) in FIG. 3, which has a sample size of 20K. As shown in FIG. 4, the phenomap power discovery system 100 utilizes a genetic association model 404 to identify a test gene target 406 from the test subset 402.

As used herein, the term “genetic association model” refers to a model that identifies associations between genetic variants and specific traits of interest (across the genome). Specifically, the phenomap power discovery system 100 uses the genetic association model 404 to analyze genetic data of a large number of patients (e.g., from the test subset 402 of genomic patient data samples) to identify patterns or markers (e.g., single nucleotide polymorphisms) that frequently show up for patients with the particular trait of interest compared with those without the particular trait of interest. To illustrate, the phenomap power discovery system 100 can use the genetic association model 404 to compare genomic patient data for the particular trait of interest with genomic patient data that lacks the particular trait of interest.

In one or more embodiments, the phenomap power discovery system 100 uses the genetic association model 404 to perform a logistic regression for binary traits by comparing the presence of a marker in patients with the trait of interest with the absence of a marker in patients without the trait of interest. Furthermore, the phenomap power discovery system 100 uses the genetic association model 404 to model the probability of a marker as a function of a genotype, while accounting for variations in age, sex, etc.

In some embodiments, the phenomap power discovery system 100 uses the genetic association model 404 to perform a linear regression for continuous traits (e.g., traits on a spectrum). Specifically, the phenomap power discovery system 100 uses the genetic association model 404 to determine the trait value as a linear function and outputs a coefficient for the association between a genotype and a trait value with a corresponding p-value.

In some embodiments, the phenomap power discovery system 100 uses the genetic association model 404 to perform a chi-square test for binary traits with comparison of allele or genotype frequencies. For example, the phenomap power discovery system 100 uses the genetic association model to compare observed versus expected frequencies of genotypes or alleles (e.g., for patient data samples with the trait of interest and without the trait of interest). Further, the phenomap power discovery system 100 uses the genetic association model to generate a p-value indicating whether the allele or genotype frequencies significantly differ.

In some embodiments, the phenomap power discovery system 100 uses the genetic association model 404 to perform a multivariate regression for multiple correlated traits. Specifically, the phenomap power discovery system 100 uses the multivariate regression in determining an association between a genotype and multiple traits (e.g., the phenomap power discovery system 100 generates p-values for individual traits and combined effects).

In one or more embodiments, the phenomap power discovery system 100 uses a machine learning model trained to process patient data samples and generate feature vectors representing the genotype data of patient data samples. Further, the phenomap power discovery system 100 uses machine learning techniques to compare the generated feature vectors (e.g., representing genotype data for the trait of interest) with control data (e.g., patient data without the trait of interest). Furthermore, based on the comparison, the phenomap power discovery system 100 generates a prediction of genotype features that are significant for the trait of interest.

The phenomap power discovery system 100 can utilize a variety of additional models. For example, the phenomap power discovery system 100 can utilize a Fisher’s Exact model, Cox proportional hazards regression, linear mixed models, multiple testing correction, and/or Bayesian models as the genetic association model 404.

As shown in FIG. 4, based on a variety of methods via the genetic association model 404, the phenomap power discovery system 100 generates the test gene target 406. As used herein, the term “test gene target” refers to a specific gene of interest identified in the test subset 402 by the phenomap power discovery system 100 using the genetic association model 404. Specifically, the phenomap power discovery system 100 uses the genetic association model 404 for a subset of the patient genomic data (e.g., the test subset 402 of genomic patient data samples). For instance, the phenomap power discovery system 100 uses the genetic association model 404 to compare different genomes of patients with the trait of interest. Further, the phenomap power discovery system 100 can compare the genomes of patients with the trait of interest with patient genomic data without the gene of interest. In doing so, the phenomap power discovery system 100 identifies a gene that is statistically significant based on the gene satisfying a threshold correlation with the trait of interest. In other words, the specific gene manifests in a statistically significant number of patient genomes for the test subset 402 of genomic patient data samples.

Moreover, FIG. 4 shows the phenomap power discovery system 100 comparing the test gene target 406 with a digital phenomap 408 (e.g., the digital phenomap 210 as generated in FIG. 2). Specifically, the phenomap power discovery system 100 identifies a feature vector of the test gene target 406 within the digital phenomap 408 and compares that feature vector with other feature vectors of additional genes in the digital phenomap 408. In doing so, the phenomap power discovery system 100 generates a test phenomap gene target 410.

As shown, the test phenomap gene target 410 would not have been noticeable from patient genomics alone. Indeed, FIG. 4 shows the phenomap power discovery system 100 identifying a gene from the sample size (A) discussed in FIG. 3. For instance, the illustration of the test phenomap gene target 410 is shown as a gene that would normally be deemed “irrelevant” by patient genomics alone (e.g., due to its low significance). However, based on comparing a feature vector of the test gene target 406 in the digital phenomap 408 with other feature vectors, the phenomap power discovery system 100 is able to identify additional significant genes for the trait of interest.

As used herein, the term “test phenomap gene target” refers to a gene identified from a digital phenomap 408 (e.g., a gene that is identified as significant based on a test subset of genomic patient data samples). In other words, the phenomap power discovery system 100 leverages the digital phenomap 408 (e.g., a first type of data) and the test subset of genomic patient data samples (e.g., a second type of data) to identify a gene of significance that is involved in a trait of interest of a trait class (e.g., is a gene target for the trait of interest and/or a target in a genetic pathway for the trait of interest).

Specifically, the phenomap power discovery system 100 compares a feature vector of the test gene target with feature vectors of genes from the digital phenomap 408 (e.g., genes that have been knocked out/perturbed in a biological cell). For instance, the phenomap power discovery system 100 determines the test gene target 406 and identifies that gene in the digital phenomap. For example, if the phenomap power discovery system 100 identifies the test gene target 406 as MECR from the test subset 402 of genomic patient data samples, the phenomap power discovery system 100 further identifies the embedding/feature vector for MECR in the digital phenomap 408. In other words, the phenomap power discovery system 100 identifies the biological cell that has been perturbed for the test gene target 406 (e.g., the test gene target 406 that was knocked out).

Moreover, the phenomap power discovery system 100 identifies another feature vector in the feature vector space of the digital phenomap that is similar to the test gene target 406 in the digital phenomap 408. Specifically, the phenomap power discovery system 100 uses a cosine similarity or a distance measure to identify another feature vector of an additional gene (or feature vectors of a plurality of genes) that are related to the test gene target 406.

Moreover, as shown in FIG. 4, the phenomap power discovery system 100 further compares the test phenomap gene target 410 with a gene target set 412. As used herein, the term “gene target set” refers to one or more genes of interest identified from a germline data set (e.g., a consolidated germline data set). For example, a gene target set includes genes of interest identified using the genetic association model 404 for a full dataset (e.g., the sample size 300d of 100K samples shown in FIG. 3). Specifically, the phenomap power discovery system 100 uses the genetic association model 404 for the full dataset (e.g., the combined set of genomic patient data) to identify one or more genes of interest that satisfy a threshold correlation with the trait of interest. In other words, the identified genes (e.g., the gene target set 412) manifest in a statistically significant number of patient genomes for an entire dataset. Accordingly, the phenomap power discovery system 100 uses the full dataset as a ground truth to determine how powerful the digital phenomap 408 is in identifying additional gene targets at smaller sample sizes relative to the full dataset.

As shown in FIG. 4, based on comparing the test phenomap gene target 410 with the gene target set 412, the phenomap power discovery system 100 generates a measure of target discovery power 414. As used herein, the term “measure of target discovery power” refers to a relative power of a digital phenomap in discovering gene targets (e.g., for a trait class). Specifically, the phenomap power discovery system 100 generates the measure of target discovery power 414 by comparing the test phenomap gene target 410 (e.g., the target identified from the digital phenomap 408 by comparing feature vectors of the test gene target 406 with other feature vectors in the digital phenomap 408) with the gene target set 412 identified from the full dataset (e.g., the combined set of genomic patient data).

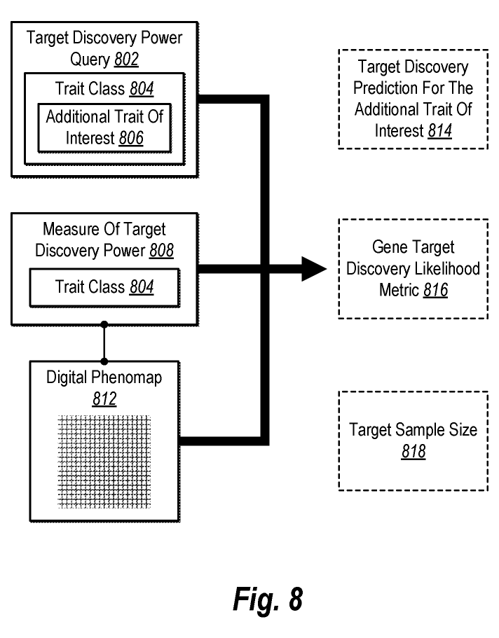

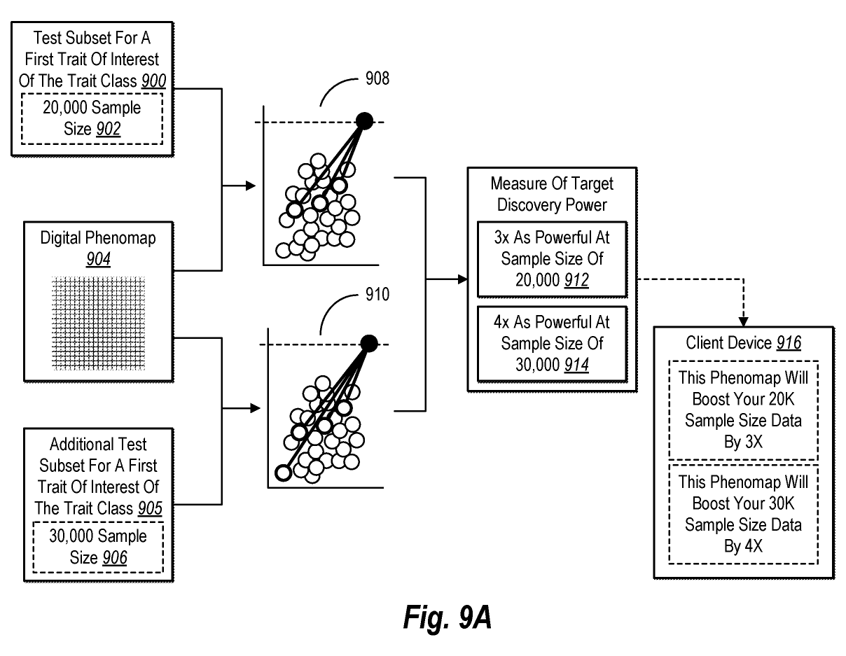

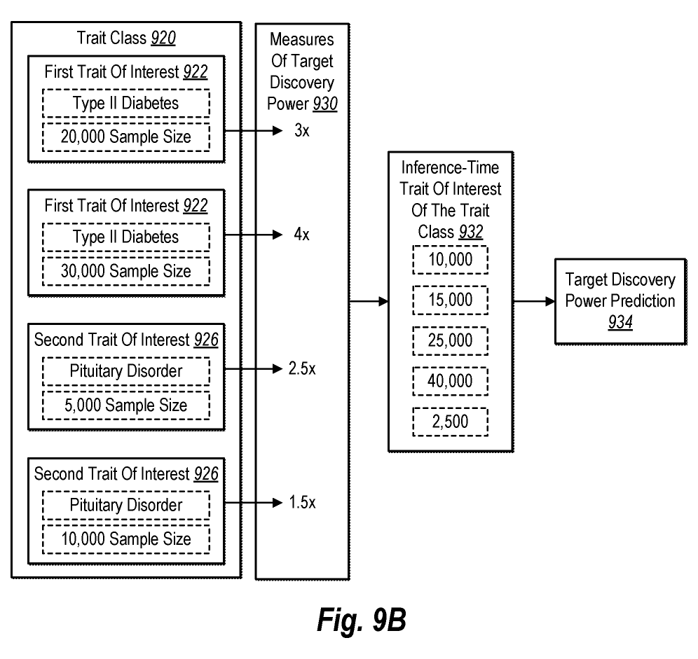

Further, the phenomap power discovery system 100 compares the test phenomap gene target 410 with the gene target set 412 (e.g., the ground truth) to determine how powerful the digital phenomap is at identifying gene targets. To illustrate, the measure of target discovery power 414 can indicate that the digital phenomap 408 boosts a dataset for a trait class by 3x. Additional detail regarding determining a measure of target discovery power is provided below (e.g., in relation to FIGS. 8-9B).

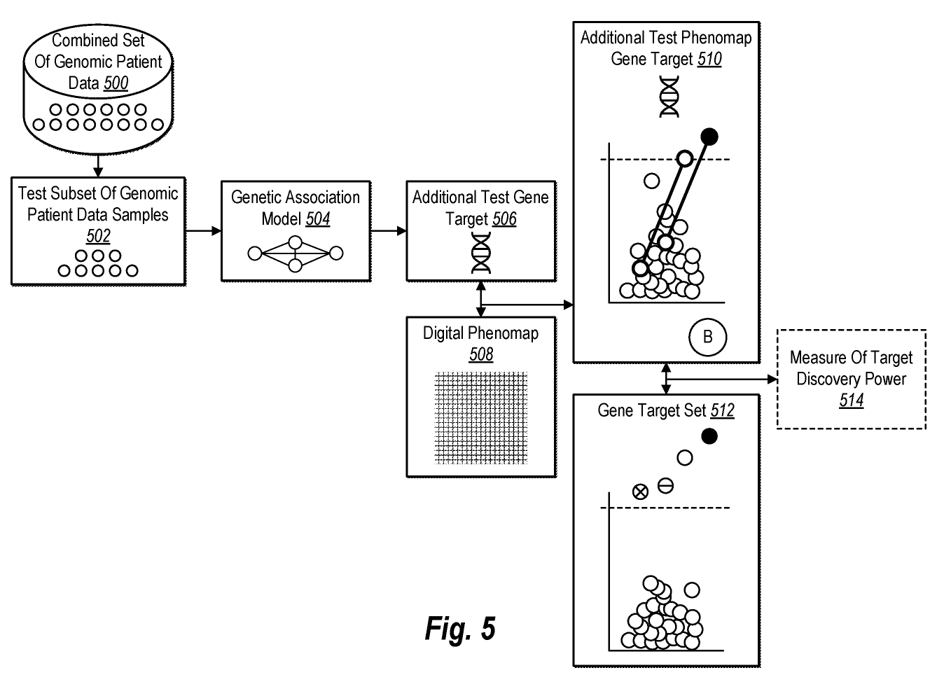

Similar to the process illustrated in FIG. 4, FIG. 5 also shows the phenomap power discovery system 100 sampling a test subset from the combined set of genomic patient data; however, FIG. 5 illustrates the phenomap power discovery system 100 sampling an increased sample size relative to the test subset in FIG. 4. For example, FIG. 5 shows the phenomap power discovery system 100 sampling an additional test subset 502 of genomic patient data samples (e.g., represented as (B) in FIG. 3) from a combined set of genomic patient data 500 (e.g., the full dataset).

As used herein, the term “additional test subset of genomic patient data samples” refers to a subset of the full dataset (e.g. ,the combined set of genomic patient data) that is a different sample size than the test subset of genomic patient data samples or it refers to a subset of the full dataset that is the same sample size but with different patient data. For example, if the test subset sample size is 20,000, then the additional test subset size can be 30,000 or the additional test subset size can be 20,000 but sampled from a different patient population (e.g., a different demographic but for the same trait of interest).

Moreover, similar to FIG. 4, FIG. 5 also illustrates the phenomap power discovery system 100 using a genetic association model 504 to generate an additional test gene target 506. As used herein, the term “additional test gene target” refers to a specific gene of interest identified by the phenomap power discovery system 100 using the genetic association model from the additional test subset 502.

As shown in FIG. 5, the phenomap power discovery system 100 identifies/generates the additional test gene target 506 in a digital phenomap 508 and compares the feature vector of the additional test gene target 506 with other feature vectors in the digital phenomap 508 to generate an additional test phenomap gene target 510. As used herein, the term “additional test phenomap gene target” refers to another test phenomap gene target that the phenomap power discovery system 100 identifies from the additional test subset 502 of genomic patient data samples (e.g., either from the same combined set of genomic patient data or another combined set of genomic patient data). Moreover, similar to FIG. 4, FIG. 5 shows the phenomap power discovery system 100 comparing the additional test phenomap gene target 510 with a gene target set 512 (e.g., the gene targets identified from the full dataset).

As shown, based on the comparison, the phenomap power discovery system 100 generates a measure of target discovery power 514. In one or more embodiments, the measure of target discovery power 514 of the digital phenomap 508 refers to a relative power of the digital phenomap 508 for discovering gene targets in a trait class (e.g., endocrinology, neurology, immunology, etc.). In some embodiments, the phenomap power discovery system 100 generates a first measure of target discovery power of the digital phenomap for a first trait of interest (e.g., type II diabetes) of a first trait class (e.g., endocrinology) and a second measure of target discovery power of the digital phenomap for a second trait of interest (e.g., pituitary disorder) of the first trait class (e.g., endocrinology).

In other words, in some embodiments, the phenomap power discovery system 100 generates sub-measures of target discovery power and combines the sub-measures to obtain the measure of target discovery power 514. To illustrate, in some embodiments, the phenomap power discovery system 100 generates a first sub-measure of target discovery power from the test subset 402 discussed in FIG. 4 and then generates a second sub-measure of target discovery power from the additional test subset 502 and further combines the first sub-measure and the second sub-measure to generate the measure of target discovery power.

As described above, the combined set of genomic patient data refers to a full dataset of genomic patient data. In other words, the combined set includes the full genomic patient data for a specific study of the trait class (e.g., type II diabetes, asthma, arthritis, blue eyes, etc.). Accordingly, an additional combined set of genomic patient data can refer to full the genomic patient data for another study of the same trait class or the full genomic patient data for another study of a different trait class.

Similar to the description above, the phenomap power discovery system 100 can utilize an additional combined set of genomic patient data to generate an additional test gene target (e.g., for an additional trait of interest of the same trait class or a different trait class). Further, the phenomap power discovery system 100 can also generate an additional test phenomap gene target from the additional test gene target. In other words, the phenomap power discovery system 100 can score the digital phenomap (e.g., the digital phenomap 408 and/or the digital phenomap 508) based on different trait classes.

Although FIGS. 4 and 5 describes the phenomap power discovery system 100 starting from a smaller sample size (20K) and moving up to a bigger sample size (30K), in one or more embodiments, the phenomap power discovery system 100 initially identifies the set of gene targets using the full dataset, and incrementally moves down in sample size relative to the full dataset. For instance, the phenomap power discovery system 100 can first sample a 30K sample size, identify the test phenomap gene target, and then sample a 20K sample size to identify another test phenomap gene target.

Furthermore, in some embodiments, the phenomap power discovery system 100 performs pre-clustering of genomic patient data to create one or more full datasets (e.g., the combined set of genomic patient data 500). Specifically, the phenomap power discovery system 100 clusters based on specific patient demographic data, patient medication data and additional electronic health records.

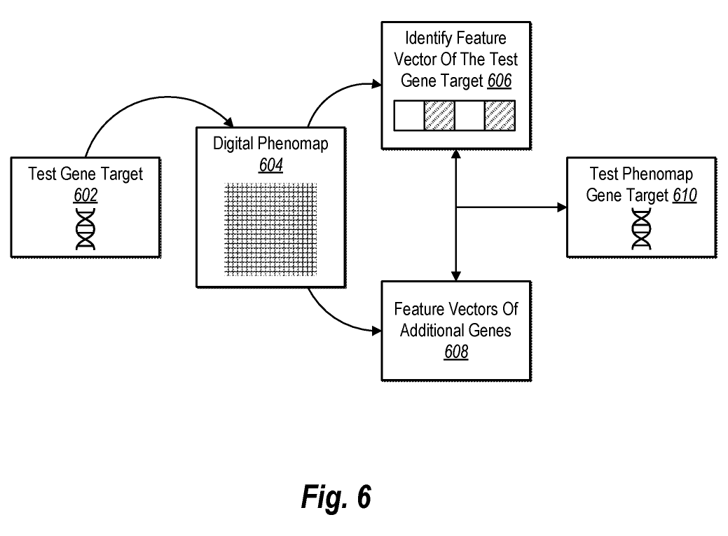

As mentioned above, the phenomap power discovery system 100 compares a feature vector of a test gene target in a digital phenomap with other feature vectors. FIG. 6, illustrates the phenomap power discovery system 100 comparing feature vectors in a digital phenomap to identify a test phenomap gene target in accordance with one or more embodiments.

As shown in FIG. 6, the phenomap power discovery system 100 identifies a test gene target 602 from using a genetic association model for a test subset of a genomic patient data samples of full dataset. In other words, the test gene target 602 is identified as being statistically significant for the test subset of a trait of interest. Moreover, the phenomap power discovery system 100 identifies the test gene target 602 (e.g., gene X) in a digital phenomap 604.

In one or more embodiments, the phenomap power discovery system 100 identifies the test gene target 602 in the digital phenomap 604 as a gene perturbation of the test gene target 602 (e.g., the test gene target 602 is knocked out in a biological cell). Specifically, the gene perturbation of the test gene target 602 in the digital phenomap 604 manifests a phenotypic trait of the biological cell based on the test gene target 602 being knocked out. In other words, the biological cell exhibits phenotypic properties from the test gene target 602 being perturbed. Moreover, the digital phenomap 604 represents the phenotypic properties by encoding a digital image of the gene knockout.

As such, FIG. 6, shows the phenomap power discovery system 100 performs an act 606 of identifying an embedding (e.g., a feature vector) of the test gene target 602 in the digital phenomap 604 and further performing an act 608 of comparing the embedding to other embeddings in the digital phenomap 604. In doing so, the phenomap power discovery system 100 identifies other perturbations (e.g., gene knockouts) that exhibit similar phenotypic properties when the test gene target 602 is knocked out in the biological cell.

In other words, the phenomap power discovery system 100 can identify genes that are part of the gene pathway or have similar manifestations to a trait of interest relative to the test gene target 602. As shown in FIG. 6, the phenomap power discovery system 100 generates a test phenomap gene target 610 from comparing the feature vector of the test gene target 602 with the other feature vectors of additional genes in the digital phenomap 604.

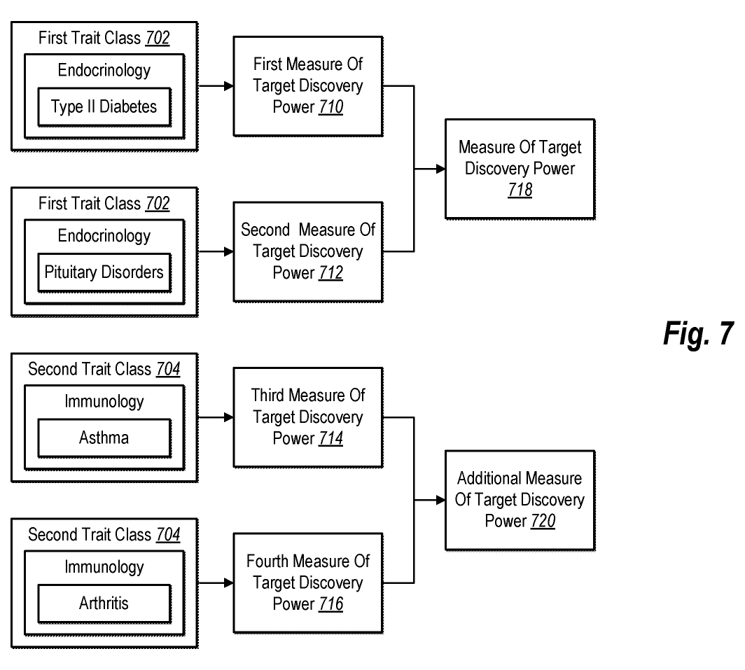

As discussed above, the phenomap power discovery system 100 can generate a measure of target discovery power for a digital phenomap relative to a trait class. FIG. 7 illustrates the phenomap power discovery system 100 generating various measures of target discovery power for a digital phenomap based on trait class and traits of interest.

As shown in FIG. 7, for a first trait class 702 (e.g., endocrinology) of a first trait of interest (e.g., type II diabetes), the phenomap power discovery system 100 generates a first measure of target discovery power 710. Further, FIG. 7 shows the phenomap power discovery system 100 generates a second measure of target discovery power 712 for the first trait class 702 (e.g., endocrinology) based on a second trait of interest (e.g., pituitary disorders).

Moreover, FIG. 7 shows that the phenomap power discovery system 100 can combine the first measure of target discovery power 710 and a second measure of target discovery power 712 to generate a measure of target discovery power 718. In other words, the phenomap power discovery system 100 generates an aggregate measure of target discovery power for the first trait class 702 based on the first trait of interest (e.g., type II diabetes) and the second trait of interest (e.g., pituitary disorders). Furthermore, the phenomap power discovery system 100 can further generate additional measures of target discovery power (e.g., third measure, fourth measure, fifth measures, etc.) for the first trait class 702 and further combine them to arrive at the measure of target discovery power 718.

As also shown in FIG. 7, the phenomap power discovery system 100 generates a third measure of target discovery power 714 for a second trait class 704 (e.g., immunology) of a third trait of interest (e.g., asthma). Further, the phenomap power discovery system 100 generates a fourth measure of target discovery power 716 for the second trait class 704 (e.g., immunology) for a fourth trait of interest (e.g., arthritis). Additionally, FIG. 7 shows the phenomap power discovery system 100 combining the third measure of target discovery power 714 and the fourth measure of target discovery power 716 to generate an additional measure of target discovery power 720. Accordingly, the phenomap power discovery system 100 generates different measures of target discovery power for different trait classes relative to a digital phenomap. Thus, a measure of target discovery power represents a relative power of the digital phenomap and how much it will boost (e.g., help discover additional gene targets) a data sample for a specific trait class.

As mentioned above, the phenomap power discovery system 100 can generate predictions for a client device at inference-time based on a digital phenomap, a measure of target discovery power for the digital phenomap, and/or genetic targets identified from samples of the inference-time trait of interest. FIG. 8 illustrates the phenomap power discovery system 100 receiving a target discovery power query for an inference-time trait of interest in accordance with one or more embodiments.

As shown in FIG. 8, the phenomap power discovery system 100 receives a target discovery power query 802. As used herein, the term “target discovery power query” refers to an inference-time query to determine the discovery power of a digital phenomap 812 relative to inference-time patient data and/or a particular trait of interest and/or to discover a measure of additional experimentation needed to identify more gene targets without using the digital phenomap 812. Specifically, the phenomap power discovery system 100 generates the measure of target discovery power of the digital phenomap 812 relative to a specific trait class (e.g., trait class 804) and at inference-time, receives the target discovery power query 802 (e.g., from a client device) for a trait of interest (e.g., additional trait of interest 806) different than the trait of interests used to generate the measure of target discovery power (e.g., Addison’s disease rather than type II diabetes).

For example, the phenomap power discovery system 100 leverages a measure of target discovery power 808 for the digital phenomap 812 to generate a target discovery power prediction 814. As used herein, the term “target discovery power prediction” refers to a prediction of the discovery power of the digital phenomap 812 for the inference-time trait of interest (e.g., Addison’s disease). Specifically, the phenomap power discovery system 100 identifies a sample size (e.g., 10K, 20K, 40K, etc.) of inference-time patient data corresponding to the target discovery power query and generates the target discovery power prediction. For example, a target discovery power prediction can include a prediction that for the inference-time trait of interest and the inference-time patient data, there is a 1.5x boost provided by the digital phenomap 812.

Moreover, FIG. 8 shows the phenomap power discovery system 100 can also generate a gene target discovery likelihood metric 816. For instance, the gene target discovery likelihood metric includes a metric for discovering additional gene targets in the digital phenomap 812 for the additional trait of interest 806 (e.g., Addison’s disease). To illustrate, the gene target discovery likelihood metric 816 can include a measure such as a probability of identifying an additional target gene by utilizing a phenomap (e.g., there is a 60% probability of finding a gene target from the phenomap). In other words, the gene target discovery likelihood metric indicates a likelihood of finding additional gene targets from the digital phenomap for the inference-time trait of interest (e.g., the additional trait of interest 806).

Furthermore, FIG. 8 shows the phenomap power discovery system 100 can generate a target sample size 818 (e.g., a sample size needed to uncover additional inference-time signals through a genetic association model or without the digital phenomap). For example, consider a circumstance where the phenomap power discovery system 100 initially has access to a genetic association (e.g., an anchor signal or some biological data that acts as an anchor) for inference time test samples. In other words, a querying device (e.g., the device that submits the target discovery power query 802) provides a genetic association test for their samples (e.g., that identifies gene targets). The phenomap power discovery system 100 can additionally provide a target sample size estimating the number of samples needed to uncover additional genetic associations (e.g., additional inference-time gene targets) utilizing a genetic association model (e.g., without utilizing the digital phenomap). Thus, the phenomap power discovery system 100 can generate a prediction for increased power of the phenomap as well as the number of additional samples needed without the phenomap to identify an additional inference-time gene target.

As used herein, the term “inference-time patient data” refers to the data provided (e.g., by a client device or server) at inference time (e.g., as part of a target discovery power query). Specifically, the inference-time patient data can include a trait of interest and/or genomic patient data. As used herein, the term “sample size” refers to a number of individual observations or units for a study, experiment, or survey. Specifically, sample size refers to a number of patient genomes for a trait of interest. For example, the sample size for a combined set of genomic patient data can be 100K while the sample size of a test subset of genomic patient data samples can be 50K.

In one or more embodiments, at inference-time, the phenomap power discovery system 100 can identify the digital phenomap 812 from a plurality of digital phenomaps with corresponding measures of target discovery power. In other words, the phenomap power discovery system 100 generates a plurality of measures of target discovery power for a plurality of digital phenomaps stored in a digital phenomap database. As discussed above, each of the measures of target discovery power can correspond to a trait class. Accordingly, at inference-time, the phenomap power discovery system 100 identifies the trait class 804, and then further identifies digital phenomaps that are measured for the trait class 804. From there, the phenomap power discovery system 100 can extract the digital phenomap 812 based the target discovery power relative to other digital phenomaps (e.g., select the phenomap with the highest measure of target discovery power).

In one or more embodiments, the phenomap power discovery system 100 can determine at inference-time, that the measures of target discovery power for a plurality of digital phenomaps fail to satisfy a threshold score. Specifically, the phenomap power discovery system 100 can determine that the measure of target discovery power 808 for the digital phenomap 812 of the trait class 804 is below threshold score and can further generate a notification to an administrator device. For instance, the phenomap power discovery system 100 can identify and prioritize the creation of a new digital phenomap for the trait class 804 based on the digital phenomap 812 being below the threshold score. In doing so, the phenomap power discovery system 100 can create more digital phenomaps that boost the data of inference-time datasets. To illustrate, the phenomap power discovery system 100 can initiate perturbation experiments to fulfill the requirements of creating a more relevant digital phenomap for the trait class 804 (e.g., a new phenomap that includes embeddings for a different cell type, different experimental assay, different digital image, different embedding model, or different type of map altogether such as a transcriptomic map, etc.).

FIG. 9A illustrates the phenomap power discovery system 100 providing a measure of target discovery power to a client device. For example, FIG. 9A illustrates the phenomap power discovery system 100 generating a measure of target discovery power based on a test subset and an additional test subset. Specifically, FIG. 9A shows the phenomap power discovery system 100 sampling a test subset 900 for a first trait of interest of a trait class from a full dataset, where a sample size 902 of the test subset 900 is 20K.

For instance, as shown, for the test subset 900 that includes the 20K sample, the phenomap power discovery system 100 identifies three test phenomap gene targets (e.g., utilizing a digital phenomap 904) from a test gene target 908 identified as satisfying a threshold correlation with the first trait of interest of the trait class. Thus, as shown, the phenomap power discovery system 100 determines that a digital phenomap used to discover the three test phenomap gene targets is 3x as powerful at the sample size 902 of 20K. Accordingly, FIG. 9A shows a first measure of target discovery power 912 for the test subset 900.

Furthermore, FIG. 9A shows the phenomap power discovery system 100 sampling an additional test subset 905 of the first trait of interest of the trait class with a sample size 906 of 30K. As shown in FIG. 9A, the phenomap power discovery system 100 identifies four test phenomap gene targets (e.g., using the digital phenomap 904) from a test gene target 910 identified as satisfying a threshold correlation with the first trait of interest of the trait class. Thus, as shown, the phenomap power discovery system 100 determines that the digital phenomap used to discover the three test phenomap gene targets is 4x as powerful at the sample size 906 of 30K. Accordingly, FIG. 9A shows a second measure of target discovery power 914 for the additional test subset 905.

Moreover, FIG. 9A shows the phenomap power discovery system 100 providing the first measure of target discovery power 912 and the second measure of target discovery power 914 to a client device 916 (e.g., the client device that submitted the target discovery power query). As such, the client device 916 can determine how powerful the digital phenomap of the phenomap power discovery system 100 is relative to inference-time patient data provided by the client device 916.

As mentioned in FIG. 8, the phenomap power discovery system 100 can generate a target discovery prediction of a relative power of the digital phenomap for inference-time patient data provided by a client device. FIG. 9B provides additional details of the phenomap power discovery system 100 generating the target discovery power prediction in accordance with one or more embodiments.

As shown in FIG. 9B, the phenomap power discovery system 100 initially generates measures of target discovery power 930 for a digital phenomap for a trait class 920. As shown in FIG. 9B, the trait class 920 can include a first trait of interest 922 (e.g., type II diabetes with a sample size of 20K), the first trait of interest 922 at a 30K sample size, a second trait of interest 926 (e.g., pituitary disorder at a 5K sample size), and the second trait of interest 926 at a 10K sample size.

Furthermore, FIG. 9B shows that the phenomap power discovery system 100 generates the measures of target discovery power 930 for each of the traits of interest. Specifically, the phenomap power discovery system 100 can generate a first measure of target discovery power of 3x for the first trait of interest 922 at the 20K sample size, a second measure of target discovery power of 4x for the first trait of interest 922 at the 30K sample size, a third measure of target discovery power of 2.5x for the second trait of interest 926 at the 5K sample size, and a fourth measure of target discovery power of 1.5x for the second trait of interest 926 at the 10K sample size.

Moreover, FIG. 9B illustrates that based on the measures of target discovery power 930, the phenomap power discovery system 100 can further generate a target discovery power prediction 934 in response to an inference-time trait of interest 932 of the trait class 920. Specifically, FIG. 9B illustrates that the phenomap power discovery system 100 can receive the inference-time trait of interest 932 at a sample size of 10K, 15K, 25K, 40K, or 2.5K samples. Based on the sample size of the inference-time patient data samples, the phenomap power discovery system 100 generates the target discovery power prediction 934.

In one or more embodiments, the phenomap power discovery system 100 generates the target discovery power prediction 934 based on combining the measures of target discovery power. For instance, for an inference-time patient data sample size that is greater than the sample size at test time (e.g., 40K sample size, the biggest sample size at test time was 30K), the phenomap power discovery system 100 can combine measures of target discovery power to estimate a power of the digital phenomap for the inference-time patient data sample size (e.g., combine a 4x boost for the 30K sample size with a 1.5x boost for the 10K sample size).

In some embodiments, the phenomap power discovery system 100 generates the target discovery power prediction 934 based on identifying the sample size that most closely resembles the inference-time patient data sample size. For instance, the phenomap power discovery system 100 identifies that the inference-time patient data sample size as 10K and further determines that the target discovery power prediction 934 is 1.5x (e.g., the same measure of target discovery power for the 10K sample size at test time).

In some embodiments, the phenomap power discovery system 100 generates the target discovery power prediction 934 based on an average of the test-time data. For instance, the phenomap power discovery system 100 identifies the inference-time patient data sample size as 15K, which is between the 10K and 20K sample size during test time. As such, the phenomap power discovery system 100 can average the 3x and the 1.5x measures of target discovery power to generate a 2.25x boost that the digital phenomap would provide to the inference-time patient data sample size of 15K.

In some embodiments, the phenomap power discovery system 100 generates the target discovery power prediction 934 based on reducing a measure of target discovery power by a threshold amount. For instance, the phenomap power discovery system 100 identifies the inference-time patient data sample size as 2,500 which is half as much as the smallest sample size during test time. As such, the phenomap power discovery system 100 can reduce the measure of target discovery power (2.5x) of the 5K sample size by half to generate a 1.25x target discovery power prediction.

In some embodiments, the phenomap power discovery system 100 leverages one or more statistical models to generate the target discovery power prediction 934. For instance, the phenomap power discovery system 100 utilizes interpolation and extrapolation to generate the target discovery power prediction 934 for an inference-time data patient samples. To illustrate, the phenomap power discovery system 100 can create a linear regression for the test time measures of target discovery power. Specifically, the linear regression can include a proportional relationship between sample size and measures of target discovery power, and the target discovery power prediction 934 of the sample size of the inference-time trait of interest would be based on the linear regression.

In some embodiments, the phenomap power discovery system 100 can create a polynomial regression to capture non-linear trends of the measures of target discovery power and determine the target discovery power prediction 934 based on the polynomial regression. In some embodiments, the phenomap power discovery system 100 can create a logarithmic or exponential model to capture the measures of target discovery power 930 of the different sample sizes. As such, the phenomap power discovery system 100 determines the target discovery power prediction 934 from the logarithmic or exponential model.

In one or more embodiments, the phenomap power discovery system 100 can utilize various techniques to arrive at the target discovery power prediction 934, such as combining (e.g., weighted averaging), scaling factors (e.g., scaling logarithmically or quadratically), smoothing techniques (e.g., Gaussian process regression to create a smooth curve), piecewise models, and machine learning models.

In some embodiments, the phenomap power discovery system 100 leverages one or more machine learning models to generate the target discovery power prediction 934. For instance, the phenomap power discovery system 100 can train a machine learning model to generalize the target discovery power prediction from training data that includes sample size and a corresponding measure of target discovery power. Specifically, the phenomap power discovery system 100 can train a random forest model, a gradient boosting model, or various types of neural networks to generate the target discovery power prediction 934.

In some implementations, the phenomap power discovery system 100 performs a pathway analysis. For instance, FIG. 9B can illustrate an implementation a pathway analysis for different traits of interest (e.g., type II diabetes and pituitary disorders) and the different traits of interest are associated with various genes in these pathways associated with the traits of interest. In other words, the phenomap power discovery system 100 can perform an analysis (e.g., using a digital phenomap) to determine the predictive power of the digital phenomap with respect to pathway(s) of traits of interest. In some embodiments, the phenomap power discovery system 100 further determines the lack of predictive power of a digital phenomap with respect to pathway(s) of traits of interest.

For example, in some embodiments, the pathway analysis is a KEGG pathway analysis, a.k.a., a Kyoto Encyclopedia of Genes and Genomes analysis. For instance, the KEGG pathway analysis can provide insight into various metabolic processes and genes involved in that process. As shown in FIG. 9B, in some embodiments, where the pathway analysis involves a KEGG pathway analysis, the phenomap power discovery system 100 can determine various measures of target discovery power for a metabolic trait class. Further, the phenomap power discovery system 100 can leverage the various measures of target discovery power for the metabolic trait class to further infer target discovery power prediction for an inference-time trait of interest of the metabolic trait class.

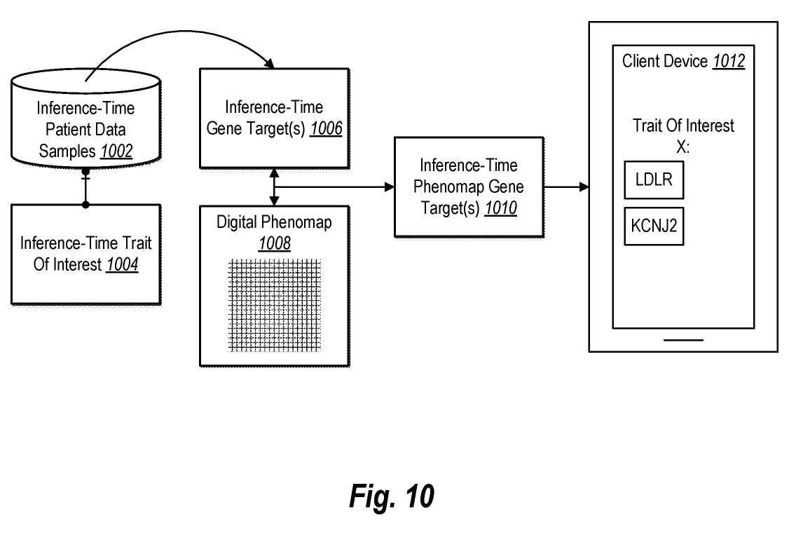

FIG. 10 illustrates the phenomap power discovery system 100 generating inference-time phenomap gene targets for inference-time patient data samples in accordance with one or more embodiments. As mentioned above, the phenomap power discovery system 100 receives a target discovery power query from a client device at inference time. Specifically, the target discovery power query relates to determining for a client device how powerful of a boost a digital phenomap would provide to inference-time patient data samples 1002.

As mentioned above, the inference-time patient data samples 1002 includes a gene list and/or genomic patient data. Specifically, FIG. 10 shows that the inference-time patient data samples 1002 are for an inference-time trait of interest 1004 of the same trait class that a digital phenomap 1008 was scored on (e.g., the phenomap power discovery system 100 generated a measure of target discovery power for the digital phenomap 1008 based on the same trait class as the inference-time trait of interest 1004). In one or more embodiments, the inference-time patient data samples 1002 can include summary statistics that do not include patient identifiers. In other words, the phenomap power discovery system 100 can process the inference-time patient data samples 1002 in an anonymized manner to protect patient privacy while still generating inference-time phenomap gene target(s) 1010.

As shown in FIG. 10, the phenomap power discovery system 100 generates inference-time gene target(s) 1006 from the inference-time patient data samples 1002. Specifically, the phenomap power discovery system 100 can use a genetic association model to generate the inference-time gene target(s) 1006. Moreover, FIG. 10 shows the phenomap power discovery system 100 comparing the inference-time gene target(s) 1006 in the digital phenomap 1008 with additional genes in the digital phenomap 1008. For instance, the phenomap power discovery system 100 finds feature vector(s) of the inference-time gene target(s) in the digital phenomap 1008 and compares them with additional feature vectors of other genes to identify genes that are close/similar to the inference-time gene target(s) 1006 (e.g., based on a distance metric or a cosine similarity measure). In doing so, the phenomap power discovery system 100 generates inference-time phenomap gene target(s) 1010 (e.g. LDLR and KCNJ2) and provides them to a client device 1012.

Although the foregoing description has focused on phenomaps (and embeddings of phenomic images), the phenomap power discovery system 100 can also operate with other maps of biology. For example, the phenomap power discovery system 100 can utilize a transcriptomic map that includes embeddings from transcriptomic profiles. To illustrate, the phenomap power discovery system 100 can apply perturbations to cells and capture counts of transcript RNAs within the perturbed cells. The phenomap power discovery system 100 can build a transcriptomic profile for a perturbation by consolidating the RNA counts for a particular perturbation across the genome. The phenomap power discovery system 100 can then utilize a model to generate an embedding of the transcriptomic profile. Moreover, the phenomap power discovery system 100 can combine transcriptomic embeddings to generate a transcriptomic map. The phenomap power discovery system 100 can also compare transcriptomic embeddings from the transcriptomic map to determine similarity measures (e.g., using cosine similarity or distance metrics). The phenomap power discovery system 100 can utilize the transcriptomic map in place of the phenomic map throughout this application.

Similarly, the phenomap power discovery system 100 can utilize other maps of biology. For example, the phenomap power discovery system 100 can generate an invivomics map (e.g., a map of embeddings representing features observed from living animals subject to a perturbation), a proteomics map, or other map of biology.

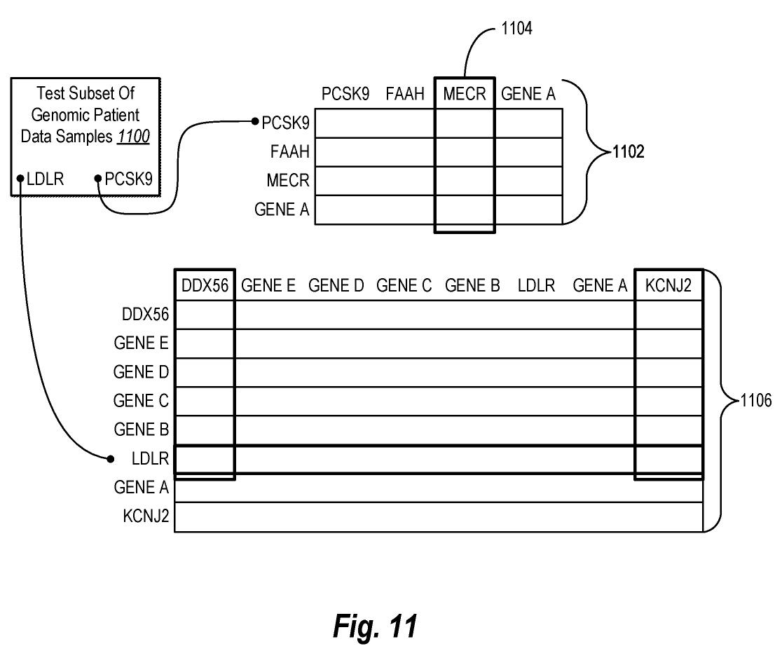

FIG. 11 illustrates experimental results of the phenomap power discovery system 100 accurately identifying genes from a digital phenomap as confirmed by set of gene targets from a full data (e.g., a combined set of genomic patient data). For example, FIG. 11 shows a test subset 1100 of genomic patient data samples. For instance, the test subset 1100 includes 24K samples from a full dataset and the phenomap power discovery system 100 utilizes a genetic association model to identify test gene targets (PCSK9 and LDLR).

Moreover, FIG. 11 shows the phenomap power discovery system 100 using a digital phenomap 1102. Specifically, FIG. 11 shows the phenomap power discovery system 100 identifying PCSK0 in the digital phenomap 1102 and further identifying a similar gene based on comparing the feature vector of PCSK9 with additional feature vectors in the digital phenomap 1102. In doing so, the phenomap power discovery system 100 identifies a test phenomap gene target 1104 (e.g., MECR).

Furthermore, in line with the principles discussed above, the phenomap power discovery system 100 analyzes a full dataset of 325K samples (e.g., the test subset 1100 was a 24K sample). In other words, the phenomap power discovery system 100 uses high-powered genetic association techniques to identify genetic relationships from relatively large datasets. For instance, the phenomap power discovery system 100 uses a genetic association model to confirm that MECR is detected in the full dataset. As such, FIG. 11 illustrates that the phenomap power discovery system 100 leverages the digital phenomap 1102 to identify significant genes at a much lower sample size (e.g., 24K samples rather than 325K samples).

Moreover, as shown, for LDLR identified from the test subset 1100 of 24K, the phenomap power discovery system 100 uses a digital phenomap 1106 to further identify the feature vector of LDLR. Specifically, the phenomap power discovery system 100 compares the feature vector of LDLR with other feature vectors of other genes. As shown, the phenomap power discovery system 100 identifies DDX56 and KCNJ2 as significant genes using the digital phenomap 1106.

Also, in line with the principles discussed above, the phenomap power discovery system 100 analyzes a full dataset (1.6 million genomic patient data samples) using a genetic association model and discovers KCNJ2 as having a significant relationship with LDLR. Further, the phenomap power discovery system 100 analyzes a full dataset (87K genomic patient data samples) to identify DDX56 as a gene having a significant relationship with LDLR. Accordingly, the phenomap power discovery system 100 uses the digital phenomap to mitigate sample size limitations of existing systems to identify known and novel genetic relationships of a trait of interest. As demonstrated in FIG. 11, the phenomap power discovery system 100 can identify the known/novel genetic relationships with a sample size as low as 24K.

Although the above description relates to generating measures of target discovery power for trait classes of germline patient data, in one or more embodiments, the phenomap power discovery system 100 can generate measures of target discovery power for trait classes related to other domains such as proteomics, vivomics, transcriptomics, and other non-germline domains.

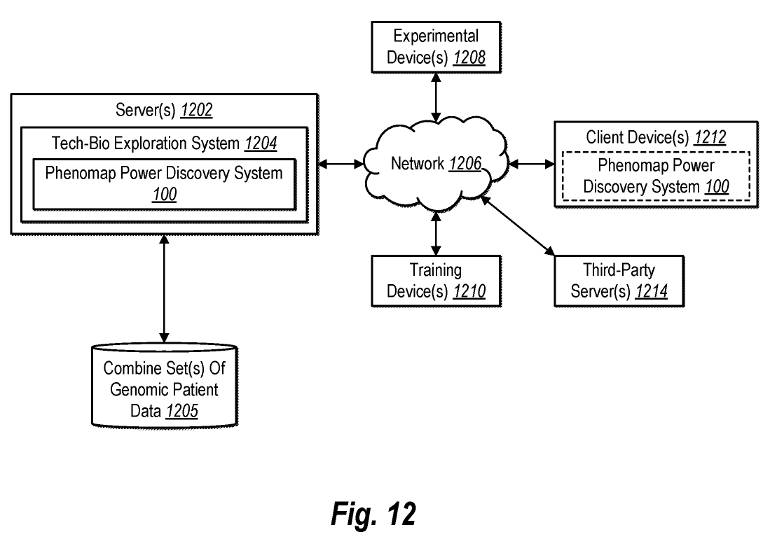

Additional details regarding the phenomap power discovery system 100 will now be provided with reference to the figures. In particular, FIG. 12 illustrates a schematic diagram of a system environment in which the phenomap power discovery system 100 can operate in accordance with one or more embodiments.

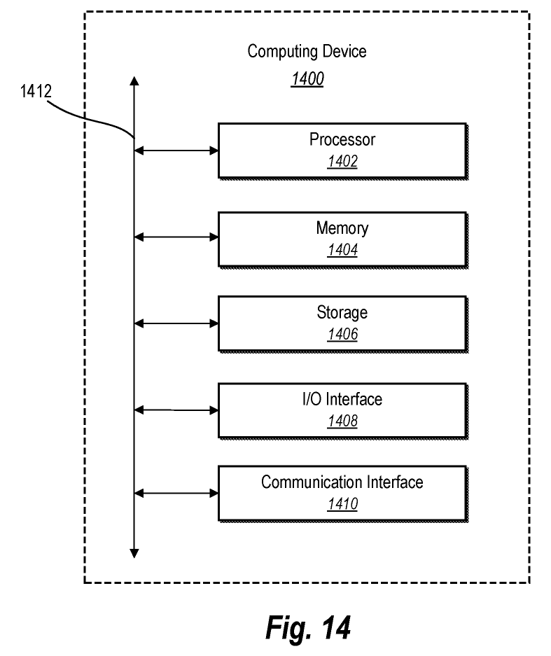

As shown in FIG. 12, the environment includes server(s) 1202 (which includes a tech-bio exploration system 1204 and the phenomap power discovery system 100), a network 1206, experimental device(s) 1208, third-party server(s) 1214, training device(s) 1210, client device(s) 1212, and combined set(s) of genomic patient data 1205. As further illustrated in FIG. 12, the various computing devices within the environment can communicate via the network 1206. Although FIG. 12 illustrates the phenomap power discovery system 100 being implemented by a particular component and/or device within the environment, the phenomap power discovery system 100 can be implemented, in whole or in part, by other computing devices and/or components in the environment (e.g., administrator device(s) / the client device(s) 1210). Additional description regarding the illustrated computing devices is provided with respect to FIG. 14 below.

As shown in FIG. 12, the server(s) 1202 (e.g., one or more local servers operated by a particular entity) can include the tech-bio exploration system 1204. In some embodiments, the tech-bio exploration system 1204 can determine, store, generate, and/or display tech-bio information including maps of biology, biology experiments from various sources, and/or machine learning tech-bio predictions. For instance, the tech-bio exploration system 1204 can analyze data signals corresponding to various treatments or interventions (e.g., compounds or biologics) and the corresponding relationships in genetics, protenomics, phenomics (i.e., cellular phenotypes), and invivomics (e.g., expressions or results within a living animal).

For instance, the tech-bio exploration system 1204 can generate and access experimental results corresponding to gene sequences, protein shapes/folding, protein/compound interactions, phenotypes resulting from various interventions or perturbations (e.g., gene knockout sequences or compound treatments), and/or invivo experimentation on various treatments in living animals. By analyzing these signals (e.g., utilizing various machine learning models), the tech-bio exploration system 1204 can generate or determine a variety of predictions and inter-relationships for improving treatments/interventions.

To illustrate, the tech-bio exploration system 1204 can generate maps of biology indicating biological inter-relationships or similarities between these various input signals to discover potential new treatments. For example, the tech-bio exploration system 1204 can utilize machine learning and/or maps of biology to identify a similarity between a first gene associated with disease treatment and a second gene previously unassociated with the disease based on a similarity in resulting phenotypes from gene knockout experiments. The tech-bio exploration system 1204 can then identify new treatments based on the gene similarity (e.g., by targeting compounds the impact the second gene). Similarly, the tech-bio exploration system 1204 can analyze signals from a variety of sources (e.g., protein interactions, or invivo experiments) to predict efficacious treatments based on various levels of biological data.

The tech-bio exploration system 1204 can generate GUIs comprising dynamic user interface elements to convey tech-bio information and receive user input for intelligently exploring tech-bio information. Indeed, as mentioned above, the tech-bio exploration system 1204 can generate GUIs displaying different maps of biology that intuitively and efficiently express complex interactions between different biological systems for identifying improved treatment solutions. Furthermore, the tech-bio exploration system 1204 can also electronically communicate tech-bio information between various computing devices.

As shown in FIG. 12, the tech-bio exploration system 1204 can include a system that facilitates various models or algorithms for generating maps of biology (e.g., maps or visualizations illustrating similarities or relationships between genes, proteins, diseases, compounds, and/or treatments) and discovering new treatment options over one or more networks. For example, the tech-bio exploration system 1204 collects, manages, and transmits data across a variety of different entities, accounts, and devices. In some cases, the tech-bio exploration system 1204 is a network system that facilitates access to (and analysis of) tech-bio information within a centralized operating system. Indeed, the tech-bio exploration system 1204 can link data from different network-based research institutions to generate and analyze maps of biology.

As shown in FIG. 12, the tech-bio exploration system 1204 can include a system that comprises the phenomap power discovery system 100 that samples from a full dataset (e.g., a combined set), identifies a test gene target (e.g., using a genetic association model), generates a test phenomap gene target, and generates a measure of target discovery power of a digital phenomap. For example, the phenomap power discovery system 100 can prepare a drug discovery pipeline by initially generating measures of target discovery power for digital phenomaps to further generate predictions/inferences of inference-time datasets. In other words, the phenomap power discovery system 100 can determine a measure of power of a digital phenomap relative to a trait class and use that to determine predictive power for gene targets for inference-time data.

As also illustrated in FIG. 12, the environment includes the client device(s) 1212. For example, the client device(s) 1212 may include, but is not limited to, a mobile device (e.g., smartphone, tablet) or other type of computing device, including those explained below with reference to FIG. 14. Additionally, the client device(s) 1212 can include a computing device associated with (and/or operated by) user accounts for the tech-bio exploration system 1204. Moreover, the environment can include various numbers of client devices that communicate and/or interact with the tech-bio exploration system 1204 and/or the phenomap power discovery system 100.

Furthermore, in one or more implementations, the client device(s) 1210 includes a client application. The client application can include instructions that (upon execution) cause the client device(s) 1210 to perform various actions. For example, a user of a user account can interact with the client application on the client device(s) 1210 to access tech-bio information, generate gene targets, generate measures of target discovery power, generate digital phenomaps, modify digital phenomaps, generate rating metrics for a gene target, generate program ratings for a trait of interest based on gene targets, generate gene target discovery likelihood metrics, generate target sample sizes, initiate training of a machine learning model utilizing a machine learning data set, and/or generate GUIs, machine learning predictions/results, and/or machine learning efficacy.

As further shown in FIG. 12, the environment includes the network 1206. As mentioned above, the network 1206 can enable communication between components of the environment. In one or more embodiments, the network 1206 may include a suitable network and may communicate using a various number of communication platforms and technologies suitable for transmitting data and/or communication signals, examples of which are described with reference to FIG. 14. Furthermore, although FIG. 12 illustrates computing devices communicating via the network 1206, the various components of the environment can communicate and/or interact via other methods (e.g., communicate directly).

As mentioned previously, in one or more implementations, the phenomap power discovery system 100 generates and accesses machine learning objects, such as results from biological assays. As shown, in FIG. 12, the phenomap power discovery system 100 can communicate with training device(s) 1210 to obtain and then store this information. For example, the tech-bio exploration system 1204 can interact with the training device(s) 1210 that include intelligent robotic devices and camera devices for generating and capturing digital images of cellular phenotypes resulting from different perturbations (e.g., genetic knockouts or compound treatments of germline patient cells) and sequencing machines. Similarly, the training device(s) 1210 can include camera devices and/or other sensors (e.g., heat or motion sensors) capturing real-time information from animals as part of invivo experimentation. The tech-bio exploration system 1204 can also interact with a variety of other training device(s) 1210 such as devices for determining, generating, or extracting gene sequences or protein information.

As shown in FIG. 12, the environment also includes a variety of computing devices (i.e., digital repository platforms) capable of storing machine learning data objects. For instance, the phenomap power discovery system 100 can store gene perturbation embeddings, genomic patient data, measures of target discovery power, gene targets, phenomap gene targets, and ground truth measures (e.g., a set of gene targets from a full dataset), on digital repository platforms for later analysis to determine whether to initiate one or more compound exploration programs (e.g., ICG or IPG).

The phenomap power discovery system 100 can determine to initiate compound exploration programs based on the measures of target discovery power/gene targets. The compound exploration programs can include industrial program generation (IPG) and industrialized compound generation (ICG). For instance, industrial program generation (IPG) includes (i) a hit selection to identify statistically strong connections in a biological map to patient-informed phenotypes, (ii) phenomic confirmation (e.g., promising actives are confirmed by automated similarity and concentration-response analytics), (iii) Trekseq confirmation (e.g., compound and gene relationships are confirmed with transcriptomics in the map background), and (iv) Structure-Activity Relationship (SAR) confidence (e.g., actives that behave as a series are identified, and an automated recommendation for expansion is identified).

ICG applies to steps subsequent to IPG. Further, in some embodiments ICG includes rapidly searching and expanding from potential hit series in the chemical space (e.g., identified at the IPG stage) and testing the potential hits with various analytical tests (e.g., SAR screens). Accordingly, in some embodiments the phenomap power discovery system 100 can initiate IPG and/or ICG in response to generating a program rating metric from the rating metric and the causal prediction.

As used herein, the term digital repository platform includes a storage device or set of storage devices (e.g., for storing digital files corresponding to machine learning data sets). In particular, a digital repository platform can include a set of storage devices at a particular location or controlled by a particular entity. Thus, for example, a digital repository platform can include a cloud service (e.g., Amazon Web Services), a local server, or a third-party server.

For example, with regard to the server(s) 1202, local servers operating the tech-bio exploration system 1204 can store machine learning data objects on various servers distributed geographically across different parts of the country or world. Further, the phenomap power discovery system 100 can interact with third-party server(s) 1214 (e.g., servers operated and owned by separate entities, such as a coordinating partner with its own biological data). The phenomap power discovery system 100 can collaborate with third parties to generate target discovery predictions for inference-time traits of interest, gene target discovery likelihood metrics, target sample sizes for inference-time traits of interest.

In addition, the phenomap power discovery system 100 can also interact with dedicated machine learning device(s). For example, the dedicated machine learning device(s) can include computing devices or virtual machines dedicated to training or implementing large-scale machine learning models. In some implementations, the phenomap power discovery system 100 can also store machine learning data objects on the dedicated machine learning device(s). For instance, the dedicated machine learning device(s) can include models each trained separately on data specific to different trait classes. For instance, the dedicated machine learning devices can be configured to generalize data relating to sample size and measures of target discovery power for specific trait classes.

Furthermore, the environment also includes the client device(s) 1212 (e.g., administrator device(s)). For example, the phenomap power discovery system 100 can utilize the client device(s) 1212 to control various functions or operations in generating measures of target discovery power, receiving target discovery power queries from client devices at inference time, generating predictions for a client device at inference time, and training/preparing genetic association models, other prediction models, responding to requests, and/or managing a compound/drug discovery pipeline. To illustrate, the client device(s) 1212 can identify assays, set up machine learning processes, determine a framework or pipeline for analyzing machine learning models, selecting storage locations in particular digital repository platforms for digital files, and/or determine access permissions to particular digital information or for initiating certain downstream programs (e.g., IPG and ICG).

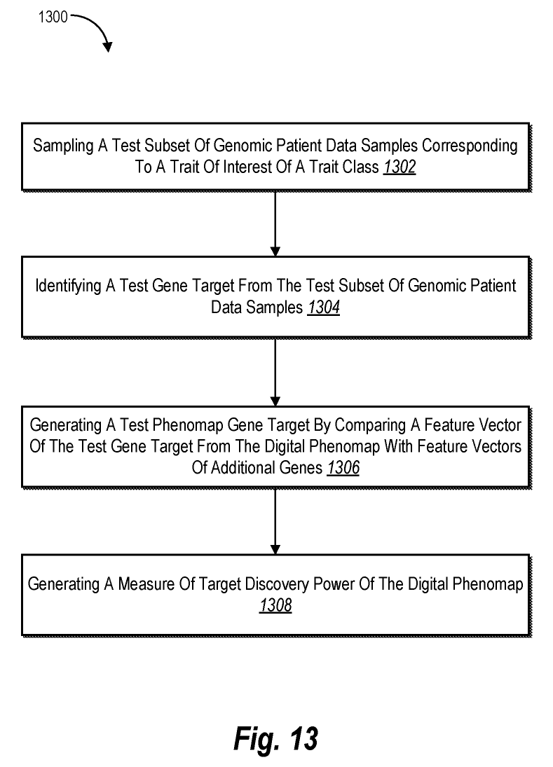

FIGS. 1-12, the corresponding text, and the examples provide a number of different systems, methods, and non-transitory computer readable media for generating measures of target discovery power for a digital phenomap. In addition to the foregoing, embodiments can also be described in terms of flowcharts comprising acts for accomplishing a particular result. For example, FIG. 13 illustrates a flowchart of an example sequence of acts in accordance with one or more embodiments.

While FIG. 13 illustrates acts according to some embodiments, alternative embodiments may omit, add to, reorder, and/or modify any of the acts shown in FIG. 13. The acts of FIG. 13 can be performed as part of a method (e.g., a computer-implemented method). Alternatively, a non-transitory computer readable medium can comprise instructions, that when executed by one or more processors (e.g., at least one processor), cause a computing device to perform the acts of FIG. 13. In still further embodiments, a system can perform the acts of FIG. 13. Additionally, the acts described herein may be repeated or performed in parallel with one another or in parallel with different instances of the same or other similar acts.

FIG. 13 illustrates an example series of acts 1300 for generating a measure of target discovery power of a digital phenomap in accordance with one or more embodiments. The series of acts 1300 can include an act 1302 of sampling a test subset of genomic patient data samples corresponding to a trait of interest of a trait class, an act 1304 of identifying a test gene target from the test subset of genomic patient data samples, an act 1306 of generating a test phenomap gene target by comparing a feature vector of the test gene target from the digital phenomap with feature vectors of additional genes, and an act 1308 of generating a measure of target discovery power of the digital phenomap. Specifically, the series of acts 1300 can include acts 1302-1308 of sampling, from a combined set of genomic patient data corresponding to a trait of interest, a test subset of genomic patient data samples corresponding to the trait of interest of a trait class; identifying, utilizing a genetic association model, a test gene target from the test subset of genomic patient data samples, wherein the test gene target satisfies a threshold correlation with the trait of interest; generating, utilizing a digital phenomap, a test phenomap gene target by comparing a feature vector of the test gene target from the digital phenomap with feature vectors of additional genes from the digital phenomap; and generating a measure of target discovery power of the digital phenomap by comparing the test phenomap gene target with a gene target set identified from the combined set of genomic patient data.

For example, in one or more embodiments, the series of acts 1300 includes receiving, from a client device, a target discovery power query for an additional trait of interest corresponding to the trait class; and providing, for display to the client device, a target discovery power prediction for the additional trait of interest based on the measure of target discovery power.

In addition, in one or more implementations, the series of acts 1300 includes identifying a sample size of inference-time patient data for the additional trait of interest corresponding to the target discovery power query; and generating the target discovery power prediction by performing at least one of: generating a gene target discovery likelihood metric for discovering additional gene targets based on the measure of target discovery power and the sample size of inference-time patient data; or generating a target sample size different than the sample size for identifying additional inference-time gene targets.

Further, in some implementations, the series of acts 1300 includes sampling, from an additional combined set of genomic patient data corresponding to an additional trait of interest of the trait class, an additional test subset of genomic patient data samples corresponding to the additional trait of interest; and identifying, utilizing the genetic association model, an additional test gene target from the additional test subset of genomic patient data samples.

In one or more implementations, the series of acts 1300 includes generating, utilizing the digital phenomap, an additional test phenomap gene target from the additional test gene target; and generating the measure of target discovery power of the digital phenomap for the trait class based on comparing the additional test phenomap gene target with an additional gene target set identified from the additional combined set of genomic patient data.

In addition, in some implementations, the series of acts 1300 includes sampling from an additional combined set of genomic patient data corresponding to an additional trait of interest, an additional test subset of genomic patient data samples corresponding to the additional trait of interest of a different trait class; identifying an additional test gene target from the additional test subset of genomic patient data samples; generating, an additional test phenomap gene target for the additional trait of interest of the different trait class; and generating an additional measure of target discovery power of the digital phenomap for the different trait class.

In one or more implementations, the series of acts 1300 includes generating a first measure of target discovery power of the digital phenomap for the trait of interest of the trait class; generating a second measure of target discovery power of the digital phenomap for an additional trait of interest of the trait class; and combining the first measure of target discovery power and the second measure of target discovery power to generate the measure of target discovery power of the digital phenomap for the trait class.

In one or more implementations, the series of acts 1300 includes sampling, from the combined set of genomic patient data, an additional test subset of genomic patient data samples corresponding to the trait of interest of the trait class, wherein the additional test subset of genomic patient data samples is for a sample size different than a sample size of the test subset of genomic patient data samples; and identifying, utilizing the genetic association model, an additional test gene target from the additional test subset of genomic patient data samples.

In one or more implementations, the series of acts 1300 includes generating, utilizing the digital phenomap, an additional test phenomap gene target by comparing a feature vector of the additional test gene target from the digital phenomap with the feature vectors of the additional genes from the digital phenomap; and generating the measure of target discovery power based on the test phenomap gene target the additional test phenomap gene target, and the gene target set identified from the combined set of genomic patient data.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., memory), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a “NIC”), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed by a processor, cause a general-purpose computer, special purpose computer, or special purpose processing device to perform a certain function or group of functions. In some embodiments, computer-executable instructions are executed by a general-purpose computer to turn the general-purpose computer into a special purpose computer implementing elements of the disclosure. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. As used herein, the term “cloud computing” refers to a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In addition, as used herein, the term “cloud-computing environment” refers to an environment in which cloud computing is employed.

FIG. 14 illustrates a block diagram of an example computing device 1400 that may be configured to perform one or more of the processes described above. One will appreciate that one or more computing devices, such as the computing device 1400 may represent the computing devices described above. In one or more embodiments, the computing device 1400 may be a mobile device (e.g., a mobile telephone, a smartphone, a PDA, a tablet, a laptop, a camera, a tracker, a watch, a wearable device, etc.). In some embodiments, the computing device 1400 may be a non-mobile device (e.g., a desktop computer or another type of client device). Further, the computing device 1400 may be a server device that includes cloud-based processing and storage capabilities.

As shown in FIG. 14, the computing device 1400 can include one or more processor(s) 1402, memory 1404, a storage device 1406, input/output interfaces 1408 (or “I/O interfaces 1408”), and a communication interface 1410, which may be communicatively coupled by way of a communication infrastructure (e.g., bus 1412). While the computing device 1400 is shown in FIG. 14, the components illustrated in FIG. 14 are not intended to be limiting. Additional or alternative components may be used in other embodiments. Furthermore, in certain embodiments, the computing device 1400 includes fewer components than those shown in FIG. 14. Components of the computing device 1400 shown in FIG. 14 will now be described in additional detail.

In particular embodiments, the processor(s) 1402 includes hardware for executing instructions, such as those making up a computer program. As an example, and not by way of limitation, to execute instructions, the processor(s) 1402 may retrieve (or fetch) the instructions from an internal register, an internal cache, memory 1404, or a storage device 1406 and decode and execute them.

The computing device 1400 includes memory 1404, which is coupled to the processor(s) 1402. The memory 1404 may be used for storing data, metadata, and programs for execution by the processor(s). The memory 1404 may include one or more of volatile and non-volatile memories, such as Random-Access Memory (“RAM”), Read-Only Memory (“ROM”), a solid-state disk (“SSD”), Flash, Phase Change Memory (“PCM”), or other types of data storage. The memory 1404 may be internal or distributed memory.

The computing device 1400 includes a storage device 1406 includes storage for storing data or instructions. As an example, and not by way of limitation, the storage device 1406 can include a non-transitory storage medium described above. The storage device 1406 may include a hard disk drive (HDD), flash memory, a Universal Serial Bus (USB) drive or a combination these or other storage devices.

As shown, the computing device 1400 includes one or more I/O interfaces 1408, which are provided to allow a user to provide input to (such as user strokes), receive output from, and otherwise transfer data to and from the computing device 1400. These I/O interfaces 1408 may include a mouse, keypad or a keyboard, a touch screen, camera, optical scanner, network interface, modem, other known I/O devices or a combination of such I/O interfaces 1408. The touch screen may be activated with a stylus or a finger.

The I/O interfaces 1408 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O interfaces 1408 are configured to provide graphical data to a display for presentation to a user. The graphical data may be representative of one or more graphical user interfaces and/or any other graphical content as may serve a particular implementation.

The computing device 1400 can further include a communication interface 1410. The communication interface 1410 can include hardware, software, or both. The communication interface 1410 provides one or more interfaces for communication (such as, for example, packet-based communication) between the computing device and one or more other computing devices or one or more networks. As an example, and not by way of limitation, communication interface 1410 may include a network interface controller (NIC) or network adapter for communicating with an Ethernet or other wire-based network or a wireless NIC (WNIC) or wireless adapter for communicating with a wireless network, such as a WI-FI. The computing device 1400 can further include a bus 1412. The bus 1412 can include hardware, software, or both that connects components of computing device 1400 to each other.

In one or more implementations, various computing devices can communicate over a computer network. This disclosure contemplates any suitable network. As an example, and not by way of limitation, one or more portions of a network may include an ad hoc network, an intranet, an extranet, a virtual private network (“VPN”), a local area network (“LAN”), a wireless LAN (“WLAN”), a wide area network (“WAN”), a wireless WAN (“WWAN”), a metropolitan area network (“MAN”), a portion of the Internet, a portion of the Public Switched Telephone Network (“PSTN”), a cellular telephone network, or a combination of two or more of these.

In particular embodiments, the computing device 1400 can include a client device that includes a requester application or a web browser, such as MICROSOFT INTERNET EXPLORER, GOOGLE CHROME, or MOZILLA FIREFOX, and may have one or more add-ons, plug-ins, or other extensions, such as TOOLBAR or YAHOO TOOLBAR. A user at the client device may enter a Uniform Resource Locator (“URL”) or other address directing the web browser to a particular server (such as server), and the web browser may generate a Hyper Text Transfer Protocol (“HTTP”) request and communicate the HTTP request to server. The server may accept the HTTP request and communicate to the client device one or more Hyper Text Markup Language (“HTML”) files responsive to the HTTP request. The client device may render a webpage based on the HTML files from the server for presentation to the user. This disclosure contemplates any suitable webpage files. As an example, and not by way of limitation, webpages may render from HTML files, Extensible Hyper Text Markup Language (“XHTML”) files, or Extensible Markup Language (“XML”) files, according to particular needs. Such pages may also execute scripts such as, for example and without limitation, those written in JAVASCRIPT, JAVA, MICROSOFT SILVERLIGHT, combinations of markup language and scripts such as AJAX (Asynchronous JAVASCRIPT and XML), and the like. Herein, reference to a webpage encompasses one or more corresponding webpage files (which a browser may use to render the webpage) and vice versa, where appropriate.

In particular embodiments, the tech-bio exploration system 1204 may include a variety of servers, sub-systems, programs, modules, logs, and data stores. In particular embodiments, the tech-bio exploration system 1204 may include one or more of the following: a web server, action logger, API-request server, transaction engine, cross-institution network interface manager, notification controller, action log, third-party-content-object-exposure log, inference module, authorization/privacy server, search module, user-interface module, user-profile (e.g., provider profile or requester profile) store, connection store, third-party content store, or location store. The tech-bio exploration system 1204 may also include suitable components such as network interfaces, security mechanisms, load balancers, failover servers, management-and-network-operations consoles, other suitable components, or any suitable combination thereof. In particular embodiments, the tech-bio exploration system 1204 may include one or more user-profile stores for storing user profiles and/or account information for credit accounts, secured accounts, secondary accounts, and other affiliated financial networking system accounts. A user profile may include, for example, biographic information, demographic information, financial information, behavioral information, social information, or other types of descriptive information, such as interests, affinities, or location.

The web server may include a mail server or other messaging functionality for receiving and routing messages between the tech-bio exploration system 1204 and one or more client devices. An action logger may be used to receive communications from a web server about a user’s actions on or off the tech-bio exploration system 1204. In conjunction with the action log, a third-party-content-object log may be maintained of user exposures to third-party-content objects. A notification controller may provide information regarding content objects to a client device. Information may be pushed to a client device as notifications, or information may be pulled from a client device responsive to a request received from the client device. Authorization servers may be used to enforce one or more privacy settings of the users of the tech-bio exploration system 1204. A privacy setting of a user determines how particular information associated with a user can be shared. The authorization server may allow users to opt in to or opt out of having their actions logged by the tech-bio exploration system 1204 or shared with other systems, such as, for example, by setting appropriate privacy settings. Third-party-content-object stores may be used to store content objects received from third parties. Location stores may be used for storing location information received from a client device associated with users.

In the foregoing specification, the invention has been described with reference to specific example embodiments thereof. Various embodiments and aspects of the invention(s) are described with reference to details discussed herein, and the accompanying drawings illustrate the various embodiments. The description above and drawings are illustrative of the invention and are not to be construed as limiting the invention. Numerous specific details are described to provide a thorough understanding of various embodiments of the present invention.

The present invention may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel to one another or in parallel to different instances of the same or similar steps/acts. The scope of the invention is, therefore, indicated by the appended claims rather than by the foregoing description. All changes that come within the meaning and range of equivalency of the claims are to be embraced within their scope.

What is claimed is:

1. A computer-implemented method comprising:
sampling, from a combined set of genomic patient data corresponding to a trait of interest, a test subset of genomic patient data samples corresponding to the trait of interest of a trait class;
identifying, utilizing a genetic association model, a test gene target from the test subset of genomic patient data samples, wherein the test gene target satisfies a threshold correlation with the trait of interest;
generating, utilizing a digital phenomap, a test phenomap gene target by comparing a feature vector of the test gene target from the digital phenomap with feature vectors of additional genes from the digital phenomap; and
generating a measure of target discovery power of the digital phenomap by comparing the test phenomap gene target with a gene target set identified from the combined set of genomic patient data.

2. The computer-implemented method of claim 1, further comprising:
receiving, from a client device, a target discovery power query for an additional trait of interest corresponding to the trait class; and
providing, for display to the client device, a target discovery power prediction for the additional trait of interest based on the measure of target discovery power.

3. The computer-implemented method of claim 2, further comprising:
identifying a sample size of inference-time patient data for the additional trait of interest corresponding to the target discovery power query; and
generating the target discovery power prediction by performing at least one of:
generating a gene target discovery likelihood metric for discovering additional gene targets based on the measure of target discovery power and the sample size of inference-time patient data; or
generating a target sample size different than the sample size for identifying additional inference-time gene targets .

4. The computer-implemented method of claim 1, further comprising:
sampling, from an additional combined set of genomic patient data corresponding to an additional trait of interest of the trait class, an additional test subset of genomic patient data samples corresponding to the additional trait of interest; and
identifying, utilizing the genetic association model, an additional test gene target from the additional test subset of genomic patient data samples.

5. The computer-implemented method of claim 4, further comprising:
generating, utilizing the digital phenomap, an additional test phenomap gene target from the additional test gene target; and
generating the measure of target discovery power of the digital phenomap for the trait class based on comparing the additional test phenomap gene target with an additional gene target set identified from the additional combined set of genomic patient data.

6. The computer-implemented method of claim 1, further comprising:
sampling from an additional combined set of genomic patient data corresponding to an additional trait of interest, an additional test subset of genomic patient data samples corresponding to the additional trait of interest of a different trait class;
identifying an additional test gene target from the additional test subset of genomic patient data samples;
generating, an additional test phenomap gene target for the additional trait of interest of the different trait class; and
generating an additional measure of target discovery power of the digital phenomap for the different trait class.

7. The computer-implemented method of claim 1, wherein generating the measure of target discovery power comprises:
generating a first measure of target discovery power of the digital phenomap for the trait of interest of the trait class;
generating a second measure of target discovery power of the digital phenomap for an additional trait of interest of the trait class; and
combining the first measure of target discovery power and the second measure of target discovery power to generate the measure of target discovery power of the digital phenomap for the trait class.

8. The computer-implemented method of claim 1, further comprising:
sampling, from the combined set of genomic patient data, an additional test subset of genomic patient data samples corresponding to the trait of interest of the trait class, wherein the additional test subset of genomic patient data samples is for a sample size different than a sample size of the test subset of genomic patient data samples; and
identifying, utilizing the genetic association model, an additional test gene target from the additional test subset of genomic patient data samples.

9. The computer-implemented method of claim 8, further comprising:
generating, utilizing the digital phenomap, an additional test phenomap gene target by comparing a feature vector of the additional test gene target from the digital phenomap with the feature vectors of the additional genes from the digital phenomap; and
generating the measure of target discovery power based on the test phenomap gene target the additional test phenomap gene target, and the gene target set identified from the combined set of genomic patient data.

10. A system comprising:
at least one processor; and
at least one non-transitory computer-readable storage medium storing instructions that, when executed by the at least one processor, cause the system to:
sample, from a combined set of genomic patient data corresponding to a trait of interest, a test subset of genomic patient data samples corresponding to the trait of interest of a trait class;
identify, utilizing a genetic association model, a test gene target from the test subset of genomic patient data samples, wherein the test gene target satisfies a threshold correlation with the trait of interest;
generate, utilizing a digital phenomap, a test phenomap gene target by comparing a feature vector of the test gene target from the digital phenomap with feature vectors of additional genes from the digital phenomap; and
generate a measure of target discovery power of the digital phenomap by comparing the test phenomap gene target with a gene target set identified from the combined set of genomic patient data.

11. The system of claim 10, further comprising instructions that, when executed by the at least one processor, cause the system to:
receive, from a client device, a target discovery power query for an additional trait of interest corresponding to the trait class; and
provide, for display to the client device, a target discovery power prediction for the additional trait of interest based on the measure of target discovery power.

12. The system of claim 11, further comprising instructions that, when executed by the at least one processor, cause the system to:
identify a sample size of inference-time patient data for the additional trait of interest corresponding to the target discovery power query; and
generate the target discovery power prediction by performing at least one of:
generating a gene target discovery likelihood metric for discovering additional gene targets based on the measure of target discovery power and the sample size of inference-time patient data; or
generating a target sample size different than the sample size for identifying additional inference-time gene targets.

13. The system of claim 10, further comprising instructions that, when executed by the at least one processor, cause the system to:
sample, from an additional combined set of genomic patient data corresponding to an additional trait of interest of the trait class, an additional test subset of genomic patient data samples corresponding to the additional trait of interest; and
identify, utilizing the genetic association model, an additional test gene target from the additional test subset of genomic patient data samples.

14. The system of claim 13, further comprising instructions that, when executed by the at least one processor, cause the system to:
generate, utilizing the digital phenomap, an additional test phenomap gene target from the additional test gene target; and
generate the measure of target discovery power of the digital phenomap for the trait class based on comparing the additional test phenomap gene target with an additional gene target set identified from the additional combined set of genomic patient data.

15. The system of claim 10, further comprising instructions that, when executed by the at least one processor, cause the system to generate the measure of target discovery power by:
generating a first measure of target discovery power of the digital phenomap for the trait of interest of the trait class;
generating a second measure of target discovery power of the digital phenomap for an additional trait of interest of the trait class; and
combining the first measure of target discovery power and the second measure of target discovery power to generate the measure of target discovery power of the digital phenomap for the trait class.

16. A non-transitory computer-readable medium storing instructions that, when executed by at least one processor, cause a computing device to:
sample, from a combined set of genomic patient data corresponding to a trait of interest, a test subset of genomic patient data samples corresponding to the trait of interest of a trait class;
identify, utilizing a genetic association model, a test gene target from the test subset of genomic patient data samples, wherein the test gene target satisfies a threshold correlation with the trait of interest;
generate, utilizing a digital phenomap, a test phenomap gene target by comparing a feature vector of the test gene target from the digital phenomap with feature vectors of additional genes from the digital phenomap; and
generate a measure of target discovery power of the digital phenomap by comparing the test phenomap gene target with a gene target set identified from the combined set of genomic patient data.

17. The non-transitory computer-readable medium of claim 16, further comprising instructions that, when executed by the at least one processor, cause the computing device to:
receive, from a client device, a target discovery power query for an additional trait of interest corresponding to the trait class; and
provide, for display to the client device, a target discovery power prediction for the additional trait of interest based on the measure of target discovery power.

18. The non-transitory computer-readable medium of claim 17, further comprising instructions that, when executed by the at least one processor, cause the computing device to:
identify a sample size of inference-time patient data for the additional trait of interest corresponding to the target discovery power query; and
generate the target discovery power prediction by performing at least one of:
generating a gene target discovery likelihood metric for discovering additional gene targets based on the measure of target discovery power and the sample size of inference-time patient data; or
generating a target sample size different than the sample size for identifying additional inference-time gene targets.

19. The non-transitory computer-readable medium of claim 16, further comprising instructions that, when executed by the at least one processor, cause the computing device to:
sample, from the combined set of genomic patient data, an additional test subset of genomic patient data samples corresponding to the trait of interest of the trait class, wherein the additional test subset of genomic patient data samples is for a sample size different than a sample size of the test subset of genomic patient data samples; and
identify, utilizing the genetic association model, an additional test gene target from the additional test subset of genomic patient data samples.

20. The non-transitory computer-readable medium of claim 19, further comprising instructions that, when executed by the at least one processor, cause the computing device to:
generate, utilizing the digital phenomap, an additional test phenomap gene target by comparing a feature vector of the additional test gene target from the digital phenomap with the feature vectors of the additional genes from the digital phenomap; and
generate the measure of target discovery power based on the test phenomap gene target the additional test phenomap gene target, and the gene target set identified from the combined set of genomic patient data.
